(19) United States
(12) Patent Application Publication
(10) Pub. No.: US 2003/0228608 A1
(43) Pub. Date: Dec. 11, 2003

(54) Method to enhance homologous recombination

(75) Inventors: Rachel Friedman-Ohana, Madison, WI (US); Michael R. Slater, Madison, WI (US); Nigel John Grinter, Buffalo Grove, IL (US)

Correspondence Address: SCHWEGMAN, LUNDBERG, WOESSNER & KLUTH, P.A., P.O. BOX 2938, MINNEAPOLIS, MN, 55402, US

(73) Assignee: Promega Corporation

(21) Appl. No.: 10/414,729

(22) Filed: Apr. 15, 2003

Related U.S. Application Data
(60) Provisional application No. 60/373,100, filed on Apr. 16, 2002.

(51) Int. Cl. C12Q 1/68; C12P 19/34

(52) U.S. Cl. 435/6; 435/91.2

(57) ABSTRACT

The invention relates to methods for enhancing the targeting of exogenous polynucleotides to a preselected target sequence in a target cell or in an extrachromosomal sequence.

### CROSS-REFERENCE TO RELATED APPLICATIONS

This application claims the benefit of the filing date of U.S. application Serial No. 60/373,100, filed Apr. 16, 2002, under 35 U.S.C. §119(e), the disclosure of which application is incorporated by reference herein.

### BACKGROUND OF THE INVENTION

Homologous recombination (or general recombination) is defined as the exchange of homologous segments anywhere along a length of two DNA molecules. An essential feature of homologous recombination is that the enzymes responsible for the recombination event can presumably use any pair of homologous sequences as substrates, although some types of sequences may be favored over others. Both genetic and cytological studies have indicated that such a crossing-over process occurs between pairs of homologous chromosomes during meiosis in higher organisms.

A primary step in homologous recombination is DNA strand exchange, which involves a pairing of a DNA duplex with at least one DNA strand containing a complementary sequence to form an intermediate recombination structure containing heteroduplex DNA (see, Radding, 1982; U.S. Pat. No. 4,888,274). The heteroduplex DNA may take several forms, including a three DNA strand containing triplex form wherein a single complementary strand invades the DNA duplex (Hsieh et al., 1990; Rao et al., 1991) and, when two complementary DNA strands pair with a DNA duplex, a classical Holliday recombination joint or chi structure (Holliday, 1964) may form, or a double-D loop (see U.S. Pat. No. 5,948,653). Once formed, a heteroduplex structure may be resolved by strand breakage and exchange, so that all or a portion of an invading DNA strand is spliced into a recipient DNA duplex, adding or replacing a segment of the recipient DNA duplex. Alternatively, a heteroduplex structure may result in gene conversion, wherein a sequence of an invading strand is transferred to a recipient DNA duplex by repair of mismatched bases using the invading strand as a template (Lewin, 1987; Lopez et al., 1987). Whether by the mechanism of breakage and rejoining or by the mechanism(s) of gene conversion, formation of heteroduplex DNA at homologously paired joints can serve to transfer genetic sequence information from one DNA molecule to another. The ability of homologous recombination (gene conversion and classical strand breakage/rejoining) to transfer genetic sequence information between DNA molecules makes targeted homologous recombination a powerful method in genetic engineering and gene manipulation.

For example, targeted recombination events can be used to correct mutations at known sites, replace genes or gene segments with defective ones, or introduce foreign genes into cells. The efficiency of such gene targeting techniques is related to several parameters: the efficiency of DNA delivery into cells, the type of DNA packaging (if any) and the size and conformation of the incoming DNA, the length and position of regions homologous to the target site (all these parameters also likely affect the ability of the incoming homologous DNA sequences to survive intracellular nuclease attack), the efficiency of hybridization and recombination and whether recombinant events are homologous or nonhomologous. While targeted homologous recombination provides a general basis for targeting and altering essentially any desired sequence in a duplex DNA molecule, targeted homologous recombination is a rare event, necessitating complex cell selection schemes to identify and isolate correctly targeted recombinants.

Several proteins or purified extracts having the property of promoting homologous recombination (i.e., recombinase activity) have been identified in prokaryotes and eukaryotes (Cox and Lehman, 1987; Radding, 1982; Madiraju et al., 1988; McCarthy et al., 1988; Lopez et al., 1987). These general recombinases presumably promote one or more steps in the formation of homologously-paired intermediates, strand-exchange, gene conversion, and/or other steps in the process of homologous recombination. In particular, the frequency of homologous recombination in prokaryotes is significantly enhanced by the presence of recombinase activities. Several purified proteins catalyze homologous pairing and/or strand exchange in vitro, including but not limited to: E. Coli RecA protein, T4 UvsX protein, Rec1 protein from Ustilago maydis, Redβ from lambda bacteriophage (Kowalczykowski et al., 1994), RecT from the cryptic Rac prophage of E. coli (Kowalczykowski et al., 1994), Rad51 protein from S. cerevisiae (Sung et al., 1994), radA from Archaeoplobus fulgidus (McIlwraith et al, 2001) and human cells (Baumann et al., 1996).

Recombinases, like the RecA protein of E. coli, are proteins that promote strand pairing and exchange. The most studied recombinase to date has been the RecA recombinase of E. coli, which is involved in homology search and strand exchange reactions (Cox and Lehman, 1987). RecA is required for induction of the SOS repair response, DNA repair, and efficient genetic recombination in E. coli. RecA can catalyze homologous pairing and strand exchange between a linear duplex DNA and a homologous single strand DNA in vitro. In contrast to site-specific recombinases, proteins like RecA which are involved in general recombination, recognize and promote pairing of DNA structures on the basis of shared homology, as has been shown by several in vitro experiments (Hsieh and Camerini-Otero, 1989; Howard-Flanders et al., 1984; Register et al., 1987). Several investigators have used RecA in vitro to promote homologously paired triplex DNA (Cheng et al., 1988; Ferrin and Camerini-Otero, 1991; Ramdas et al., 1989; Strobel et al., 1991; Hsieh et al., 1990; Rigas et al., 1986), and Pati et al. (U.S. Pat. No. 5,948,653) employed purified RecA in a method for targeted homologous recombination in prokaryotic and eukaryotic cells.

Nevertheless, there exists a need in the art for increasing the efficiency of targeted homologous recombination.

### SUMMARY OF THE INVENTION

The invention provides methods for targeting an at least partially single stranded nucleic acid substrate for recombination to a preselected target nucleic acid sequence. The at least partially single stranded substrate of the invention comprises two exogenous nucleic acid molecules comprising targeting polynucleotides which substantially correspond to or are substantially complementary to the preselected target nucleic acid sequence, and the two nucleic acid molecules are capable of forming a partially double stranded molecule with each other. In the presence of recombinase, the targeting polynucleotides localize (or target) to one or more preselected target nucleic acid sequence(s) by homologous pairing (e.g., in vitro with an extrachromosomal sequence, or in vivo with an extrachromosomal sequence or chromosomal DNA) to form a recombination intermediate. The resolution of the recombination intermediate in vivo yields a targeted sequence alteration (e.g., an insertion, deletion, substitution, or any combination thereof) with high efficiency and sequence specificity.

In one embodiment of the invention, the nucleic acid molecules of the substrate comprise only targeting polynucleotides. Thus, the targeting polynucleotides, which substantially correspond to or are substantially complementary to the preselected target nucleic acid sequence, have one or more nucleotide alterations, for example, one or more insertions, deletions, substitutions, or any combination thereof, relative to the preselected target nucleic acid sequence. In other embodiments, the nucleic acid molecules of the substrate comprise numerous nucleotides in addition to the targeting polynucleotide, for example, a nucleic acid fragment of interest, which does not substantially correspond to nor is substantially complementary to the preselected target nucleic acid sequence.

The at least partially single stranded nature of the substrate may be the result of at least one 5′ end or one 3′ end of one of the nucleic acid molecules comprising a nucleotide sequence, the substantial complement of which is not present at the 3′ end or 5′ end, respectively, of the other nucleic acid molecule of the substrate. Thus, if the two nucleic molecules were base paired, the substrate comprises a 5′ or 3′ staggered end (protruding overhang). Preferred substrates have two staggered ends, e.g., a substrate comprising two 5′ staggered ends, a substrate comprising a 5′ and a 3′ staggered end, or a substrate comprising two 3′ staggered ends. Recombinase may be mixed with a substrate of the invention which is fully single stranded, i.e., the two nucleic acid molecules are denatured, or partially single stranded and partially double stranded. The partially single stranded nature of the substrate may also be the result of the unwinding of at least one free end of a double stranded DNA, e.g., using helicase, yielding a molecule which is partially single stranded and partially double stranded. In this embodiment, recombinase is mixed with the substrate after the formation of single stranded ends.

As described hereinbelow, the efficiency of targeted homologous recombination in E. coli with either E. coli RecA or Thermotoga maritima RecA, a plasmid target and a partially single stranded DNA (ssDNA) substrate with 5′ staggered ends was greater than that with a corresponding denatured double stranded DNA (dsDNA) substrate (i.e., the nucleic acid molecules of the dsDNA substrate are entirely complementary). The efficiency of targeted homologous recombination with a partially ssDNA substrate with 3′ staggered ends was similar to that of the corresponding denatured ssDNA substrate. It is envisioned that the efficiency of recombination with a partially single stranded substrate having 3′ staggered ends may be enhanced. For example, after intermediate formation, by adding a polymerase, e.g., T4 polymerase, DNA polymerase I, or Klenow fragment along with dNTPs to a mixture of the substrate and a target DNA, i.e., plasmid, which was grown in a dut ung host. The 3′ end of the substrate is extended by the polymerase using the target DNA as a template. The resulting product is either digested with uracil DNA glycosylase, which removes uracil bases from the DNA leaving abasic sites, and then transformed into bacteria, or simply transformed into a Dut⁺ Ung⁺ host bacteria without prior treatment by uracil DNA glycosylase. Thus, the parental strand of the target that served as the template for the DNA polymerase is degraded in the bacteria, favoring the formation of the targeted sequence alteration (see Kunkel, 1985). Alternatively, the target DNA is grown in a host that methylates newly synthesized DNA, and the 3′ end of the substrate is extended in the presence of non-methylated nucleotides. Optionally, the extended product is treated with ligase. The extended product is digested with an endonuclease that cleaves at methylated residues, e.g., DpnI, to form single-stranded nicks in the target DNA. The DNA is then transformed into the bacterial host, and the parental strand of the target that served as the template for the DNA polymerase is degraded in the bacteria, favoring the formation of the targeted sequence alteration (see U.S. Pat. No. 5,789,166, and Papworth et al., 1996).

Thus, the invention provides a method for targeting and altering, by homologous recombination, a preselected target nucleic acid sequence in an extrachromosomal sequence or in a cell, i.e., in the chromosome or an extrachromosomal sequence present in the cell. The method comprises providing a mixture comprising recombinase and an at least partially single stranded nucleic acid substrate for recombination comprising two nucleic acid molecules. The first and the second nucleic acid molecules each comprise targeting polynucleotides that substantially correspond to or are substantially complementary to the preselected target nucleic acid sequence. The two nucleic acid molecules are capable of forming a partially double stranded molecule with each other, and, in one embodiment, at least the 5′ end or the 3′ end of the first nucleic acid molecule comprises a nucleotide sequence, the substantial complement of which is not present at the 3′ end or 5′ end, respectively, of the second nucleic acid molecule, which nucleotide sequence is capable of binding recombinase. In one embodiment, the single stranded portion of the nucleic acid substrate is coated with recombinase. In one embodiment, the recombinase is a species of prokaryotic recombinase. In one embodiment, the prokaryotic recombinase is a species of prokaryotic RecA protein, e.g., E. coli RecA or Thermotoga RecA, Redβ, RecT or RadA. In another embodiment, the recombinase is a species of eukaryotic recombinase, e.g., the recombinase is Rad51 recombinase, or a complex of recombinase proteins.

In one embodiment, at least one of the nucleic acid molecules further comprises a nucleic acid fragment of interest which does not substantially correspond to or is not substantially complementary to the preselected target nucleic acid sequence. In one embodiment, at least one of the nucleic acid molecules comprises a deletion of at least one nucleotide relative to the preselected target nucleic acid sequence. In another embodiment, at least one of the nucleic acid molecules comprises a substitution of at least one nucleotide relative to the preselected target nucleic acid sequence. In a further embodiment, at least one of the nucleic acid molecules comprises an addition of at least one nucleotide relative to the preselected target nucleic acid sequence. In yet a further embodiment, at least one of the nucleic acid molecules further comprises a chemical substituent, e.g., one which is covalently attached to the nucleic acid molecule. In one embodiment, the sequence of at least one of the nucleic acid molecules comprises a deletion in a gene, promoter, intron, enhancer, open reading frame, or exon relative to the preselected target nucleic acid sequence. In a further embodiment, the sequence of at least one of the nucleic acid molecules comprises an insertion in a gene, promoter, intron, enhancer, open reading frame, or exon relative to the preselected target nucleic acid sequence. In a further embodiment, the sequence of at least one of the nucleic acid molecules comprises a substitution in a gene, promoter, intron, enhancer, open reading frame, or exon relative to the preselected target nucleic acid sequence.

The invention further provides a method for targeting and altering, by homologous recombination, a preselected target nucleic acid sequence in an extrachromosomal sequence. The method comprises providing a mixture comprising recombinase and a nucleic acid substrate for recombination comprising two nucleic acid molecules which together form a substantially double stranded molecule having single stranded 5′ and 3′ ends, wherein the first and the second nucleic acid molecules each comprise targeting polynucleotides which substantially correspond to or are substantially complementary to the preselected target nucleic acid sequence, and wherein at least one of the single stranded ends is capable of binding recombinase. The mixture is contacted with the extrachromosomal sequence to form a recombination intermediate and the recombination intermediate introduced into a cell to yield an altered cell comprising a genetically altered extrachromosomal sequence comprising a targeted sequence alteration. In one embodiment, the single stranded portion of the nucleic acid substrate is coated with recombinase. In one embodiment, the recombinase is a species of prokaryotic recombinase. In one embodiment, the prokaryotic recombinase is a species of prokaryotic RecA protein, e.g., RecA protein is E. coli RecA or Thermotoga RecA, Redβ, RecT or RadA. In another embodiment, the recombinase is a species of eukaryotic recombinase, e.g., the recombinase is Rad51 recombinase, or a complex of recombinase proteins.

In one embodiment, at least one of the nucleic acid molecules further comprises a nucleic acid fragment of interest which does not substantially correspond to or is not substantially complementary to the preselected target nucleic acid sequence. In one embodiment, at least one of the nucleic acid molecules comprises a deletion of at least one nucleotide relative to the preselected target nucleic acid sequence. In another embodiment, at least one of the nucleic acid molecules comprises a substitution of at least one nucleotide relative to the preselected target nucleic acid sequence. In a further embodiment, at least one of the nucleic acid molecules comprises an addition of at least one nucleotide relative to the preselected target nucleic acid sequence. In yet a further embodiment, at least one of the nucleic acid molecules further comprises a chemical substituent, e.g., one which is covalently attached to the nucleic acid molecule. In one embodiment, the sequence of at least one of the nucleic acid molecules comprises a deletion in a gene, promoter, intron, enhancer, open reading frame, or exon relative to the preselected target nucleic acid sequence. In a further embodiment, the sequence of at least one of the nucleic acid molecules comprises an insertion in a gene, promoter, intron, enhancer, open reading frame, or exon relative to the preselected target nucleic acid sequence. In a further embodiment, the sequence of at least one of the nucleic acid molecules comprises a substitution in a gene, promoter, intron, enhancer, open reading frame, or exon relative to the preselected target nucleic acid sequence.

In one preferred embodiment of the invention, the method comprises adding to an extrachromosomal sequence which comprises a preselected target nucleic acid sequence, at least one recombinase and at least a partially single stranded nucleic acid substrate for recombination which comprises a nucleic acid molecule comprising targeting polynucleotides so as to form a recombination intermediate comprising the extrachromosomal sequence and the nucleic acid molecules. The in vitro formed recombination intermediate is then introduced to an appropriate host cell, either a prokaryotic or eukaryotic cell, e.g., a mutant E. coli host, which resolves of the recombination intermediate between the targeting polynucleotides in at least one of the nucleic acid molecules and the preselected target nucleic acid sequence in the extrachromosomal sequence occurs. The resolution of the recombination intermediate yields a genetically altered extrachromosomal sequence comprising a targeted sequence alteration. As discussed above, this alteration may be one or more insertions, deletions or substitutions of nucleotides. In another embodiment, at least one recombinase and at least a partially single stranded nucleic acid substrate is added to a host cell, the genome of which comprises the preselected target nucleic acid sequence, i.e., the target nucleic acid sequence is in an extrachromosomal sequence or the chromosome of the cell. In this embodiment, the recombination intermediate is formed, and resolved, in vivo, yielding a targeted sequence alteration. The substrate may be introduced to the cell simultaneously or sequentially with the one or more recombinase species, and optionally with an extrachromosomal sequence comprising a preselected target nucleic acid sequence. Preferably, a host cell comprising the targeted sequence alteration is then identified and/or isolated, optionally in the absence of selection. The identification may be via sequence specific screening for the targeted sequence alteration, e.g., by the gain or loss of a restriction endonuclease site, DNA hybridization, SSCV, PCR or sequence analysis. In one embodiment, the host cell is a prokaryotic cell. In another embodiment, the host cell is a eukaryotic cell. In one embodiment, at least one of the nucleic acid molecules further comprises a nucleic acid fragment of interest which does not substantially correspond to or is not substantially complementary to the preselected target nucleic acid sequence. For example, the nucleic acid fragment of interest may be greater than 1000 nucleotides in length. In one embodiment, the nucleic acid fragment of interest comprises a gene, promoter, intron, enhancer, open reading frame, or exon which is not present in the preselected target nucleic acid sequence. The invention also further comprises identifying an altered cell having the targeted sequence alteration.

Targeted homologous recombination may be used: (1) to facilitate cloning, e.g., in prokaryotes, (2) to target chemical substituents in a sequence-specific manner, (3) to correct or to generate genetic mutations, such as base substitutions, additions, and/or deletions in genomic DNA sequences by homologous recombination and/or gene conversion, e.g., converting a mutant DNA sequence that encodes a non-functional, dysfunctional, and/or truncated polypeptide into a corrected DNA sequence that encodes a functional polypeptide (e.g., has a biological activity such as an enzymatic activity, hormone function, or other biological property), remove or create a genetic lesion in non-coding sequences (e.g., promoters, enhancers, silencers, originals of replication, or splicing signals), including methods for correcting disease alleles involved in inherited genetic diseases (e.g., cystic fibrosis) and neoplasia (e.g., neoplasms induced by somatic mutation of an oncogene or tumor suppressor gene, such as p53, or viral genes associated with neoplasia, such as HBV genes), (4) to produce homologously targeted transgenic (recombinant) organisms, including bacteria, animals and plants at high efficiency, (5) in other applications (e.g., targeted drug delivery) based on in vivo homologous pairing, (6) domain swapping, and (7) gene fusions (e.g., reporter constructs).

The use of the methods of the invention provides the general advantages of DNA manipulation via homologous recombination, e.g., precise and specific exchange of genetic information including orientation and crossover control, and precise alteration at the single base pair level regardless of the size of the substrate DNA, modification at any position of interest. Further, when the method of the invention is employed to clone DNA in, preferably, but not limited to, prokaryotic cells, the method has the additional advantages of avoiding the use of processes or enzymes necessary for techniques currently known in the art (i.e., restriction enzymes, ligase, phosphatase and site-specific recombinases for cloning and gene modification). The method also has advantages for rapid directional cloning without gel purification, high yields of desired recombinant DNA without selection (e.g., 10-20%), and single-base control in fusing the sequence in the targeting polynucleotide to the preselected target DNA, e.g., without employing site-specific recombination sites or restriction endonuclease sites. Moreover, using the methods of the invention, larger insertions of DNA can be accomplished than previously reported, i.e., insertions of 100 kb or more can be achieved by this method. In particular, insertions which are greater in size (polynucleotide length) than the size of the sum of targeting polynucleotides, can be achieved.

A plurality of substrates of the invention comprising a library of mismatches between the targeting nucleotides and the target nucleic acid sequence is useful to generate a library of variant nucleic acid sequences of a preselected target nucleic acid sequence, e.g., a target nucleic acid sequence in an extrachromosomal sequence in vitro or in vivo, or in a chromosome. As employed herein, “mismatches” includes one or more substitutions, insertions and/or deletions in a sequence, i.e., the mismatched sequence is a variant sequence relative to the sequence in a reference sequence, e.g., the target sequence. A library, as used herein, includes two or more nucleic acid molecules or cells having nucleic acid sequences that have one or more mismatches relative to each other. In one embodiment, the method comprises adding to an extrachromosomal sequence comprising the preselected target nucleic sequence in vitro, recombinase and a plurality of nucleic acid substrates for recombination, to form a library of variant nucleic acid sequences. In another embodiment, the method comprises introducing into a population of cells comprising the preselected target nucleic acid substrate, recombinase and a plurality of nucleic acid substrates for recombination, to form a cellular library of variant nucleic acid sequences. Each substrate comprises two nucleic acid molecules, each molecule comprising targeting polynucleotides that substantially correspond to or are substantially complementary to the preselected target nucleic acid sequence, and the two nucleic acid molecules of a substrate are capable of forming at least a partially double stranded molecule with each other. At least one of the nucleic acid molecules comprises a single stranded nucleotide sequence that is capable of binding recombinase. For example, to prepare a plurality of substrates comprising a library of mismatches, two or more structurally and/or functionally related polynucleotides having one or more mismatches are randomly nicked by limited treatment with an endonuclease, such as DNase I. The endonuclease treated molecules are mixed, denatured and slowly cooled, yielding a population comprising a plurality of substrates for recombination which comprise at least one single stranded end capable of binding recombinase. Thus, a library of nucleic acid comprising mismatches in a portion of an open reading frame of a gene or an entire gene, or a portion of genes or entire genes from a multigene family, may be used to prepare substrates in the methods of the invention. The resulting library of sequences may be introduced into cells to form a library of genetically altered cells comprising variant nucleic acid sequences, which variant sequences may be cloned or otherwise isolated, e.g., via an amplification reaction or based on functional differences such as positive or negative selection. In one embodiment, the cells are prokaryotic cells. In another embodiment, the cells are eukaryotic cells.

In one embodiment, the invention provides a method of generating a library of recombination intermediates comprising variant nucleic acid sequences of a preselected target nucleic acid sequence in an extrachromosomal sequence. The method comprises adding to the extrachromosomal sequence, recombinase and a plurality of nucleic acid substrates for recombination, to form a library of recombination intermediates. Each substrate comprises two variant nucleic acid molecules which together form a substantially double stranded molecule having single stranded 5′ and 3′ ends, wherein the first and the second variant nucleic acid molecules each comprise targeting polynucleotides which substantially correspond to or are substantially complementary to the preselected target nucleic acid sequence. At least one of the single stranded ends is capable of binding recombinase. The plurality of substrates comprise a library of mismatches between the targeting polynucleotides and the target nucleic acid sequence. In one embodiment, the cells are prokaryotic cells. In another embodiment, the cells are eukaryotic cells.

Further provided is a method of generating a library of variant nucleic acid sequences of a preselected target nucleic acid sequence in a cell. The method comprises introducing into a population of target cells, recombinase and a plurality of at least partially single stranded nucleic acid substrates for recombination, to form a library of variant nucleic acid sequences. Each substrate comprises two nucleic acid molecules, wherein the first and the second nucleic acid molecules each comprise targeting polynucleotides which substantially correspond to or are substantially complementary to the preselected target nucleic acid sequence, wherein the two nucleic acid molecules are capable of forming a partially double stranded molecule with each other, wherein at least the 5′ end or the 3′ end of the first nucleic acid molecule comprises a nucleotide sequence, the substantial complement of which is not present at the 3′ end or 5′ end of the second nucleic acid molecule, which nucleotide sequence is capable of binding recombinase. The plurality of substrates comprise a library of mismatches between the targeting polynucleotide and the target nucleic acid sequence. In one embodiment, the cells are prokaryotic cells. In another embodiment, the cells are eukaryotic cells.

Also provided is a method of generating a library of variant nucleic acid sequences of a preselected target nucleic acid sequence in a cell, in which recombinase and a plurality of nucleic acid substrates for recombination are introduced into a population of target cells to form a library of variant nucleic acid sequences. Each substrate comprises two nucleic acid molecules which together form a substantially double stranded molecule having single stranded 5′ and 3′ ends, wherein the first and the second nucleic acid molecules each comprise targeting polynucleotides which substantially correspond to or are substantially complementary to the preselected target nucleic acid sequence, and wherein at least one of the single stranded ends is capable of binding recombinase. The plurality of substrates comprise a library of mismatches between the targeting polynucleotide and the target nucleic acid sequence. In one embodiment, the cells are prokaryotic cells. In another embodiment, the cells are eukaryotic cells.

In another embodiment, the invention provides a method of generating a library of genetically altered cells comprising variant nucleic acid sequences of a preselected target nucleic acid sequence in an extrachromosomal sequence. The method comprises adding to the extrachromosomal sequence, recombinase and a plurality of at least partially single stranded nucleic acid substrates for recombination, to form a plurality of recombination intermediates. Each substrate comprises two nucleic acid molecules, wherein the first and the second nucleic acid molecules each comprise targeting polynucleotides which substantially correspond to or are substantially complementary to the preselected target nucleic acid sequence, wherein the two nucleic acid molecules are capable of forming a partially double stranded molecule with each other, wherein at least the 5′ end or the 3′ end of the first nucleic acid molecule comprises a nucleotide sequence, the complement of which is not present at the 3′ end or 5′ end of the second nucleic acid molecule, which nucleotide sequence is capable of binding recombinase, and wherein the plurality of substrates comprise a library of mismatches between the targeting polynucleotides and the target nucleic acid sequence. The plurality of recombination intermediates is introduced into a population of cells to form a library of genetically altered cells comprising variant nucleic acid sequences. In one embodiment, the cells are prokaryotic cells. In another embodiment, the cells are eukaryotic cells.

In yet another embodiment the invention provides a method of generating a library of genetically altered cells comprising variant nucleic acid sequences of a preselected target nucleic acid sequence in an extrachromosomal sequence. The method comprises adding to the extrachromosomal sequence, recombinase and a plurality of nucleic acid substrates for recombination, to form a plurality of recombination intermediates, wherein each substrate comprises two nucleic acid molecules which together form a substantially double stranded molecule having single stranded 5′ and 3′ ends, wherein the first and the second nucleic acid molecules each comprise targeting polynucleotides which substantially correspond to or are substantially complementary to the preselected target nucleic acid sequence, wherein at least one of the single stranded ends is capable of binding recombinase, and wherein the plurality of substrates comprise a library of mismatches between the targeting polynucleotides and the target nucleic acid sequence. The plurality of recombination intermediates is introduced into a population of cells to form a library of genetically altered cells comprising variant nucleic acid sequences. In one embodiment, the cells are prokaryotic cells. In another embodiment, the cells are eukaryotic cells.

Also provided is a method of generating a library of genetically altered cells comprising variant nucleic acid sequences of a preselected target nucleic acid sequence. The method includes introducing into a population of cells comprising a preselected target nucleic acid sequence, recombinase and a plurality of at least partially single stranded nucleic acid substrates for recombination, to form a library of genetically altered cells comprising variant nucleic acid sequences. Each substrate comprises two nucleic acid molecules, wherein the first and the second nucleic acid molecules each comprise targeting polynucleotides which substantially correspond to or are substantially complementary to the preselected target nucleic acid sequence, wherein the two nucleic acid molecules are capable of forming a partially double stranded molecule with each other, and wherein at least the 5′ end or the 3′ end of the first nucleic acid molecule comprises a nucleotide sequence, the complement of which is not present at the 3′ end or 5′ end of the second nucleic acid molecule, which nucleotide sequence is capable of binding recombinase, and wherein the plurality of substrates comprise a library of mismatches between the targeting polynucleotide and the target nucleic acid sequence. In one embodiment, the cells are prokaryotic cells. In another embodiment, the cells are eukaryotic cells.

Further provided is a method of generating a library of genetically altered cells comprising variant nucleic acid sequences of a preselected target nucleic acid sequence. The method includes introducing into a population of cells comprising a preselected target nucleic acid sequence, recombinase and a plurality of nucleic acid substrates for recombination, to form a library of genetically altered cells comprising variant nucleic acid sequences. Each substrate comprises two nucleic acid molecules which together form a substantially double stranded molecule having single stranded 5′ and 3′ ends, wherein the first and the second nucleic acid molecules each comprise targeting polynucleotides which substantially correspond to or are substantially complementary to the preselected target nucleic acid sequence, wherein at least one of the single stranded ends is capable of binding recombinase, and wherein the plurality of substrates comprise a library of mismatches between the targeting polynucleotide and the target nucleic acid sequence. I one embodiment, the cells are prokaryotic cells. In another embodiment, the cells are eukaryotic cells.

In yet another embodiment, genomic DNA from one organism, e.g., one species of bacteria, is treated so as to yield a plurality of substrates with at least one single stranded end capable of binding recombinase, e.g., substrates are formed by randomly nicking genomic DNA with limited DNase I treatment, heating the treated DNA, then slowly cooling the DNA. The library of partially single stranded nucleic acid substrates is then introduced into the cells of another organism, e.g., a different species of bacteria, to form a cellular library. The library is then optionally screened for genetically altered cells having a property that is different than that of the corresponding nongenetically altered cell.

### BRIEF DESCRIPTION OF THE FIGURES

FIG. 1A. Nucleoprotein assembly over time with a fluorescein-labeled 91-mer and Thermotoga RecA or E. coli RecA.

FIG. 1B. Graph of nucleoprotein assembly over time with a fluorescein-labeled 35-mer, 51-mer or 91-mer and Thermotoga RecA or E. coli RecA.

FIG. 2A. A schematic of exemplary substrates of the invention.

FIG. 2B. A schematic of the preparation of partially ssDNA substrates of the invention having 3′ staggered ends.

FIG. 2C. A schematic of the preparation of partially ssDNA substrates of the invention having 5′ staggered ends.

FIG. 3. Percent of recombinants obtained after transformation of E. coli with nucleoprotein complexes comprising one of two different RecAs and a denatured dsDNA substrate, a partially ssDNA substrate with 5′ staggered ends, and a partially single stranded DNA substrate with 3′ staggered ends.

FIG. 4. A summary of the recombination frequencies obtained with three different substrates shown in FIG. 3.

FIG. 5. Analysis of the stability of RecA-free intermediates.

FIG. 6A. Percent of recombinants obtained after E. coli transformation with recombination intermediates comprising one of two different RecAs and a denatured dsDNA substrate comprising a tetR or a neoR gene insertion. Proteinase K treatment of samples prior to transformation decreased the number of recombinants.

FIG. 6B. Percent of recombinants obtained after E. coli transformation with recombination intermediates comprising one of two different RecAs and a dsDNA substrate comprising a tetR or a neoR gene insertion and a partially ssDNA substrate with 5′ staggered ends and a target dsDNA plasmid.

### DETAILED DESCRIPTION OF THE INVENTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

By “nucleic acid”, “oligonucleotide”, and “polynucleotide” or grammatical equivalents herein means at least two nucleotides covalently linked together. A nucleic acid of the present invention will generally contain phosphodiester bonds, although in some cases nucleic acid analogs are included that may have alternate backbones, comprising, for example, phosphoramide (Beaucage et al., 1993; Letsinger, 1970; Sprinzl et al., 1977; Letsinger et al., 1984; Letsinger et al., 1988; and Pauwels et al., 1986), phosphorothioate, phosphorodithioate, O-methylphophoroamidite linkages, and peptide nucleic acid backbones and linkages (Egholm, 1992; Meier et al., 1992; Nielsen, 1993; Carlsson et al., 1996). These modifications of the ribose-phosphate backbone or bases may be done to facilitate the addition of other moieties such as chemical constituents, including 2′ O-methyl and 5′ modified substituents, or to increase the stability and half-life of such molecules in physiological environments.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al. (1996) and Yoon et al. (1996).

In general, the nucleic acid molecules comprising targeting polynucleotides may comprise any number of structures, as long as the structures do not substantially affect the functional ability of the targeting polynucleotide to result in homologous recombination.

As used herein, the terms “predetermined” or “preselected” target DNA sequence refers to polynucleotide sequences in an isolated extrachromosomal sequence or contained in a target cell which include, for example, chromosomal sequences (e.g., structural genes, regulatory sequences including promoters and enhancers, recombinatorial hotspots, repeat sequences, integrated proviral sequences, hairpins, and palindromes), or extrachromosomal sequences (e.g., replicable plasmids or viral replication intermediates) including chloroplast and mitochondrial DNA sequences. By “predetermined” or “preselected” it is meant that the target sequence may be selected at the discretion of the practitioner on the basis of known or predicted sequence information, and is not constrained to specific sites recognized by certain site-specific recombinases (e.g., FLP recombinase or CRE recombinase). In some embodiments, the preselected DNA target sequence will be other than a naturally occurring DNA sequence (e.g., a transgene, parasitic, mycoplasmal or viral sequence). An exogenous nucleic acid molecule is a polynucleotide which is transferred into a target cell but which has not been replicated in that host cell; however, replicated copies of the polynucleotide subsequently made in the cell are endogenous sequences (and may, for example, become integrated into a cell chromosome). Similarly, transgenes that are microinjected or transfected into a cell are exogenous polynucleotides, however integrated and/or replicated copies of the transgene(s) are endogenous sequences.

The term “corresponds to” is used herein to mean that a polynucleotide sequence is homologous (i.e., may be similar or identical, not strictly evolutionarily related) to all or a portion of a reference polynucleotide sequence. In contradistinction, the term “complementary to” is used herein to mean that the complementary polynucleotide sequence is able to hybridize to the other strand. As outlined below, preferably, the homology between the two sequences is at least 70%, preferably 85%, and more preferably 95%, identical. Thus, the complementarity between two single stranded nucleic acid molecules comprising targeting polynucleotides and between targeting polynucleotides and the target nucleic acid sequence need not be perfect. For illustration, the nucleotide sequence “TATAC” corresponds to a reference sequence “TATAC” and is perfectly complementary to a reference sequence “GTATA”.

The terms “substantially corresponds to” or “substantial identity” or “homologous” as used herein denotes a characteristic of a nucleic acid sequence, wherein a nucleic acid sequence has at least about 70% sequence identity as compared to a reference sequence, typically at least about 85% sequence identity, and preferably at least about 95% sequence identity, as compared to a reference sequence. The reference sequence may be a subset of a larger sequence, such as a portion of a gene or flanking sequence, or a repetitive portion of a chromosome. However, the reference sequence is at least 20 nucleotides long, typically at least about 30 nucleotides long, and preferably at least about 50 to 100 nucleotides long. “Substantially complementary” as used herein refers to a sequence that is complementary to a sequence that substantially corresponds to a reference sequence. In general, targeting efficiency increases with the length of the targeting polynucleotide portion that is substantially complementary to a reference sequence present in the target DNA.

“Specific hybridization” is defined herein as the formation of hybrids between a targeting polynucleotide (e.g., a polynucleotide of the invention which may include substitutions, deletion, and/or additions as compared to the preselected target DNA sequence) and a selected target DNA sequence, wherein the targeting polynucleotide preferentially hybridizes to the preselected target DNA sequence such that, for example, at least one discrete band can be identified on a Southern blot of DNA prepared from target cells that contain the target DNA sequence, and/or a targeting polynucleotide in an intact cell or nucleus localizes to a discrete location. For organisms whose complete genome sequence is known, a unique target DNA sequence and targeting polynucleotide can be modeled using computer software. In some instances, a target sequence may be present in more than one target polynucleotide species (e.g., a particular target sequence may occur in multiple members of a gene family or in a known repetitive sequence). It is evident that optimal hybridization conditions will vary depending upon the sequence composition and length(s) of the targeting polynucleotide(s) and target(s), and the experimental method selected by the practitioner. Various guidelines may be used to select appropriate hybridization conditions (see, Maniatis et al., 1989 and Berger and Kimmel, 1987).

The term “naturally-occurring” as used herein as applied to an object refers to the fact that an object can be found in nature. For example, a polynucleotide sequence that is present in an organism (including viruses) that can be isolated from a source in nature and which has not been intentionally modified by man, for example, in the laboratory, is naturally-occurring.

As used herein, the term “disease allele” refers to an allele of a gene that is capable of producing a recognizable disease. A disease allele may be dominant or recessive and may produce disease directly or when present in combination with a specific genetic background or pre-existing pathological condition. A disease allele may be present in the gene pool or may be generated de novo in an individual by somatic mutation. For example, disease alleles include: activated oncogenes, a sickle cell anemia allele, a Tay-Sachs allele, a cystic fibrosis allele, a Lesch-Nyhan allele, a retinoblastoma-susceptibility allele, a Fabry's disease allele, and a Huntington's chorea allele. As used herein, a disease allele encompasses both alleles associated with human diseases and alleles associated with recognized veterinary diseases.

As used herein, the term “cell-uptake component” refers to an agent which, when bound, either directly or indirectly, to a nucleic acid molecule, e.g., enhances the intracellular uptake of the nucleic acid molecule, e.g., into at least one cell type. A cell-uptake component may include, but is not limited to, the following: specific cell surface receptors such as a galactose-terminal (asialo-) glycoprotein capable of being internalized into hepatocytes via a hepatocyte asialoglycoprotein receptor, a polycation (e.g., poly-L-lysine), and/or a protein-lipid complex formed with the nucleic acid molecule. Those of skill in the art know various combinations of the above, as well as alternative cell-uptake components.

Generally, the nomenclature used hereafter and the laboratory procedures in cell culture, molecular genetics, and nucleic acid chemistry and hybridization described below are those well known and commonly employed in the art. Standard techniques are used for recombinant nucleic acid methods, polynucleotide synthesis, cell culture, and transgenesis. Generally enzymatic reactions, oligonucleotide synthesis, oligonucleotide modification, and purification steps are performed according to the manufacturer's specifications. The techniques and procedures are generally performed according to conventional methods in the art and various general references which are provided throughout this document. The procedures therein are believed to be well known in the art and are provided for the convenience of the reader. All the information contained therein is incorporated herein by reference.

Nucleic Acid Molecules Comprising Targeting Polynucleotides

Nucleic acid molecules comprising targeting polynucleotides may be produced by chemical synthesis of oligonucleotides, polymerase chain reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, BACs, cosmids, bacteriophage DNA, other viral DNA or replication intermediates, or purified restriction fragments thereof, as well as other sources of single and double stranded polynucleotides having a desired nucleotide sequence.

Targeting polynucleotides are generally at least about 2 to 100 nucleotides long, preferably at least about 5 to about 100 nucleotides long, more preferably at least about 20 to about 200 nucleotides long, e.g., at least about 50 to 500 nucleotides long, or 2000 nucleotides, or longer; however, as the length of a nucleic acid molecule increases beyond about 20,000 to 50,000 to 400,000 nucleotides, the efficiency of transferring an intact nucleic acid molecule into the cell may decrease. The length of the targeting polynucleotide may be selected at the discretion of the practitioner on the basis of the sequence composition and complexity of the preselected target DNA sequence(s) and guidance provided in the art (Hasty et al., 1991, and Shulman et al., 1990). In a preferred embodiment, the length of the targeting polynucleotide relative to the nucleic acid molecule is from about 0.00001, 0.0001, 0.001, 0.01 or 0.1 up to 100%, but maybe from about 1 to about 20% or from about 1 to about 10%.

Targeting polynucleotides have at least one sequence that substantially corresponds to, or is substantially complementary to, a preselected target DNA sequence (i.e., a DNA sequence of a polynucleotide located in a target cell, such as a chromosomal, mitochondrial, chloroplast, viral, episomal, or mycoplasmal polynucleotide, or a DNA sequence in an exogenous (isolated) extrachromosomal sequence). Such targeting polynucleotide sequences serve as substrates for homologous pairing with the preselected target DNA sequence(s). Targeting polynucleotides are typically located at or near the 5′ end, 3′ end, internally, 5′ and 3′ end, or any combination thereof, of a nucleic acid molecule of the invention and preferably, the targeting polynucleotides are included in at least a portion of a single stranded portion of the substrate for recombination, which portion is capable of binding recombinase. Single stranded regions which are capable of binding recombinase at a level or in an amount useful to target substantially complementary sequences, are preferably at least about 20, and preferably greater than 20 nucleotides in length. The addition of recombinases to single stranded regions of the substrate for recombination which include targeting polynucleotides likely enhances the efficiency of homologous recombination between homologous sequences. The addition of recombinases also likely permits efficient gene targeting with targeting polynucleotides having short (about 20 nucleotides long) segments of homology, as well as with targeting polynucleotides having longer segments of homology.

It is preferred that targeting polynucleotides have sequences that are highly homologous to the preselected target DNA sequence(s). Typically, targeting polynucleotides of the invention have at least one region of homology that is at least about 12 to 35 nucleotides long, and it is preferable that the homology is at least about 20 to 100 nucleotides long, and more preferably at least about 50 to 500 nucleotides long, although the degree of sequence homology between the targeting polynucleotides and the targeted sequence and the base composition of the targeted sequence determines the optimal and minimal homology lengths (e.g., G-C rich sequences are typically more thermodynamically stable and generally require shorter length). Therefore, both homology length and the degree of sequence homology can only be determined with reference to a particular preselected target sequence, but homology generally must be at least about 12 nucleotides long and must also substantially correspond or be substantially complementary to a preselected target sequence. Preferably, the homology is at least about 12, and preferably at least about 22 nucleotides, more preferably at least 50 nucleotides, long and is identical to or complementary to a preselected target DNA sequence.

The formation of heteroduplex joints is not a stringent process; genetic evidence supports the view that the classical phenomena of meiotic gene conversion and aberrant meiotic segregation result in part from the inclusion of mismatched base pairs in heteroduplex joints, and the subsequent correction of some of these mismatched base pairs before replication. Observations on RecA protein have provided information on parameters that affect the discrimination of relatedness from perfect or near-perfect homology and that affect the inclusion of mismatched base pairs in heteroduplex joints. The ability of RecA protein to drive strand exchange past all single base-pair mismatches and to form extensively mismatched joints in superhelical DNA reflect its role in recombination and gene conversion. This error-prone process may also be related to its role in mutagenesis. RecA-mediated pairing reactions involving DNA of phi X174 and G4, which are about 70 percent homologous, have yielded homologous recombinants (Cunningham et al., 1981), although RecA preferentially forms homologous joints between highly homologous sequences, and likely mediates a homology search process between an invading DNA strand and a recipient DNA strand, producing relatively stable heteroduplexes at regions of high homology. Accordingly, recombinases can drive the homologous recombination reaction between strands which are significantly, but not perfectly, homologous, which allows gene conversion and the modification of target sequences. Thus, a substrate of the invention which comprises a nucleic acid molecule comprising targeting polynucleotides may be used to introduce one or more nucleotide substitutions, insertions and/or deletions into a preselected target DNA sequence, and any corresponding amino acid substitutions, insertions and deletions in proteins encoded by the altered (targeted) DNA sequence.

In one preferred embodiment, the method employs a substrate comprising two nucleic acid molecules, each molecule comprising targeting polynucleotides which substantially correspond to or are substantially complementary to the preselected target sequence, wherein each of the nucleic acid molecules has a 5′ or 3′ end, the sequence of which does not have a complementary sequence at the 3′ or 5′ end, respectively, of the other nucleic acid molecule. Preferably, the substrate, prior to contacting with recombinase or introduction into a cell, is partially double stranded (due to the complementary nature of at least the targeting polynucleotides). The substrate is incubated with RecA, another recombinase or a plurality of recombinases, so as to form a nucleoprotein complex. This complex may be mixed with an extrachromosomal sequence to form a recombination intermediate prior to introduction into a target cell or introduced directly into cells. In one embodiment, the cells are prokaryotic cells, e.g., E. coli cells.

Alternatively, a denatured form of a substrate comprising two nucleic acid molecules, each molecule comprising targeting polynucleotides which substantially correspond to or are substantially complementary to the preselected target sequence, wherein at least one of the nucleic acid molecules has a 5′ or 3′ end, the sequence of which does not have a complementary sequence at the respective 3′ or 5′ end of the other nucleic acid molecule, is incubated with at least one recombinase to form a nucleoprotein complex. As described above, this complex may be mixed with an extrachromosomal sequence to form a recombination intermediate prior to introduction into a target cell or introduced directly into cells.

The substrate and the recombinase, may be individually, sequentially, or consecutively, introduced to cells or mixed with an extrachromosomal sequence and introduced to cells. The single stranded portions of the substrate may contain a sequence that enhances the loading process of a recombinase, for example a RecA loading sequence is the recombinogenic nucleation sequence poly[d(A-C)], and its complement, poly[d(G-T)]. The duplex sequence poly[d(A-C)-d(G-T)[n], where n is from 5 to 25.

There appears to be a fundamental difference in the stability of RecA-protein-mediated D-loops formed between one single stranded DNA (ssDNA) probe hybridized to negatively supercoiled DNA targets in comparison to relaxed or linear duplex DNA targets. Internally located double stranded DNA (dsDNA) target sequences on relaxed or linear DNA targets hybridized to ssDNA probes produce single D-loops, which are unstable after removal of RecA protein (Adzuma, 1992; Hsieh et al, 1992; and Chiu et al., 1993). This DNA instability of hybrids formed with linear duplex DNA targets is most probably due to the incoming ssDNA probe W-C base pairing with the complementary DNA strand of the duplex target and disrupting the base pairing in the other DNA strand. The required high free-energy of maintaining a displaced DNA strand in an unpaired ssDNA conformation in a protein-free single D-loop apparently can be compensated for either by the stored free energy inherent in negatively supercoiled DNA targets, by the addition of a second complementary ssDNA or by base pairing initiated at the distal ends of the joint DNA molecule, allowing the exchanged strands to freely intertwine. The addition of a second RecA-coated complementary ssDNA to the three-strand containing single D-loop stabilizes hybrid joints located away from the free ends of the duplex target DNA through formation of a double D-loop (Pati et al., 1997). However, as described in the Examples below, the structure of the recombination intermediate was found to be unstable after protease digestion. Thus, the double D-loop is not a structure present in an intermediate of the invention.

Recombinase Proteins

Recombinases are proteins that, when included with nucleic acid molecules comprising targeting polynucleotides, provide a measurable increase in the recombination frequency and/or localization frequency between the targeting polynucleotide and a preselected target DNA sequence by cooperatively binding to DNA and promoting homologous pairing and DNA strand exchange between homologous DNA molecules.

In the present invention, recombinase refers to a family of RecA-like recombination proteins having essentially all or most of the same functions, particularly: (i) the ability of the recombinase to properly bind to and position targeting polynucleotides on their homologous targets and (ii) the ability of recombinase/targeting polynucleotide complexes to efficiently find and bind to complementary target sequences. Recombinases within the scope of the invention include those obtained from natural sources, i.e., cells with a wild-type recombinase, or recombinantly-produced recombinases, e.g., mutant or chimeric recombinases, including recombinases with enhanced activities relative to a corresponding naturally occurring recombinase.

The best characterized RecA protein is from E. coli, in addition to the wild-type protein, a number of mutant RecA-like proteins have been identified (e.g., RecA803; see Madiraju et al., 1988; Madiraju et al, 1992; Lavery et al., 1992; and Kowalczykowski et al., 1994). Further, many organisms have RecA-like recombinases with strand-transfer activities (e.g., Fugisawa et al., 1985; Hsieh et al., 1986; Hsieh et al., 1989; Fishel et al., 1988; Cassuto et al., 1987; Ganea et al., 1987; Moore et al., 1990; Keene et al., 1984; Kimeic, 1984; Kmeic, 1986; Kolodner et al., 1987; Sugino et al., 1985; Halbrook et al., 1989; Eisen et al., 1988; McCarthy et al., 1988; Lowenhaupt et al., 1989). Examples of such recombinase proteins include, but are not limited to RecA, RecA803, UvsX, and other RecA mutants and RecA-like recombinases (Roca, 1990), SepI (Kolodner et al., 1987; Tishkoff et al.), DST2, KEMI, XRN1 (Dykstra et al., 1991), STP alpha/DST1 (Clark et al., 1991), HPP-1 (Moore et al., 1991), other target recombinases (Bishop et al., 1992 and Shinohara et al., 1992) and RadA, e.g., from archael organisms such as Archaeoglobus fulgidus (McIlwriath et al., 2001). Other examples include RecT (Kowalczykowski et al., 1994) and Redβ (Kowalczykowski et al., 1994). RecA may be purified from E. coli strains, other bacterial strains, e.g., Thermotoga maritima, or eukaryotic cells. Some strains contain the RecA coding sequences on a “runaway” replicating plasmid vector present at a high copy numbers per cell. The RecA803 protein is a high-activity mutant of wild-type RecA. The art teaches several examples of recombinase proteins, for example, from Drosophila, yeast, plant, human, and non-human mammalian cells, including proteins with biological properties similar to RecA (i.e., RecA-like recombinases), such as Rad51 from mammals and yeast, and Pk-rec (Rashid et al., 1997). In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli RecA803 mutant with enhanced recombinase activity, and chimeric sequences comprising recombinase sequences operably linked to non-recombinase sequences or to recombinase sequences from a different source.

In a preferred embodiment, RecA or Rad51 is used. For example, RecA protein is typically obtained from bacterial strains that overproduce the protein: wild-type E. coli RecA protein and mutant RecA803 protein may be purified from such strains. Alternatively, RecA protein can also be purchased from, for example, Amersham Biosciences (Piscataway, N.J.).

RecA protein and its homologs, when coating a ssDNA, form a nucleoprotein complex. In this nucleoprotein complex, one monomer of RecA protein is bound to about 2.5 to 3 nucleotides. This property of RecA to coat ssDNA is essentially sequence independent, although particular sequences may favor initial loading of RecA onto a polynucleotide (e.g., nucleation sequences). The nucleoprotein complex(es) can be formed on essentially any DNA molecule and can be formed in cells.

Recombinase Coating of Substrates of the Invention

The conditions used to coat nucleic acid substrates with recombinases such as RecA protein and ATPγS have been described in, for example, U.S. Pat. No. 5,273,881, U.S. Pat. No. 5,223,414, or U.S. Pat. No. 5,948,653, as well as in the examples below. The examples below are directed to the use of E. coli or Thermotoga RecA, although as will be appreciated by those in the art, other recombinases may be used as well. Nucleic acid substrates can be coated using GTPγS, mixes of ATPγS with rATP, rGTP and/or dATP, or dATP or rATP alone in the presence of an rATP generating system (Sigma). Various mixtures of GTPγS, ATPγS, ATP, ADP, dATP and/or rATP or other nucleosides may be used, particularly preferred are mixes of ATPγS and dATP.

RecA protein coating of nucleic acid substrates is typically carried out as described below or in U.S. Pat. No. 5,273,881 or U.S. Pat. No. 5,948,653. Briefly, the substrate, whether fully or partially single stranded, is added to standard RecA coating reaction buffer containing ATPγS and dATP, at 42° C. (E. coli RecA) or 65° C. to 75° C. (Thermotoga RecA), and to this is added the RecA protein. Alternatively, the coating reaction may be conducted at other temperatures, e.g., 30° C. or 37° C. Alternatively, RecA protein may be included with the buffer components and ATPγS and dATP before the substrate is added.

RecA protein coating of substrate is normally carried out in a standard IX RecA coating reaction buffer. RecA protein concentrations in coating reactions vary depending upon substrate size and amount.

The coating of substrate with RecA protein can be evaluated in a number of ways. First, protein binding to DNA can be examined using band-shift gel assays (see Menthe et al., 1981 and Example 1). Labeled polynucleotides can be coated with RecA protein in the presence of ATPγS and the products of the coating reactions separated by gel electrophoresis. Following incubation of RecA protein with substrate, the RecA protein effectively coats single stranded regions. As the ratio of RecA protein monomers to nucleotides in the substrate increases, the electrophoretic mobility of the substrate decreases, i.e., is retarded, due to RecA-binding. Retardation of the mobility of the coated substrate reflects the degree of saturation of substrate with RecA protein. An excess of RecA monomers to DNA nucleotides is required for efficient RecA coating of short substrates (Leahy et al., 1986).

A second method for evaluating protein binding to DNA is in the use of nitrocellulose filter binding assays (Leahy et al., 1986 and Woodbury, et al., 1983). The nitrocellulose filter binding method is particularly useful in determining the dissociation-rates for protein:DNA complexes using labeled DNA. In the filter binding assay, DNA:protein complexes are retained on a filter while free DNA passes through the filter. This assay method is more quantitative for dissociation-rate determinations because the separation of DNA:protein complexes from free targeting polynucleotide is very rapid.

Cell-Uptake Components

A nucleic acid molecule of the invention may optionally be conjugated, typically by covalent or preferably noncovalent binding, to a cell-uptake component. Various methods have been described in the art for targeting DNA to specific cell types. A nucleic acid molecule of the invention can be conjugated to essentially any of several cell-uptake components known in the art. In one aspect of the invention, a substrate having at least one associated recombinase is targeted to cultured cells in vitro or to eukaryotic cells in vivo (i.e., in an intact animal) by exploiting the advantages of a receptor-mediated uptake mechanism, such as an asialoglycoprotein receptor-mediated uptake process. In this variation, a nucleic acid molecule comprising a targeting polynucleotide is associated with a recombinase and a cell-uptake component which enhances the uptake of the nucleic acid molecule into cells of at least one cell type in an intact individual. For example, a cell-uptake component typically consists of: (1) a galactose-terminal (asialo-) glycoprotein (e.g., asialoorosomucoid) capable of being recognized and internalized by specialized receptors (asialoglycoprotein receptors) on hepatocytes in vivo, and (2) a polycation, such as poly-L-lysine or polyethylenimine (PEI), which binds to the nucleic acid molecule, usually by electrostatic interaction.

For targeting to hepatocytes, a nucleic acid molecule can be conjugated to an asialoorosomucoid (ASOR)-poly-L-lysine conjugate by methods described in the art and incorporated herein by reference (Wu and Wu, 1987; Wu and Wu, 1988a; Wu and Wu, 1988b; Wu and Wu, 1992; Wu et al., 1991; and Wilson et al., 1992; WO 92/06180; WO 92/05250; and WO 91/17761).

Alternatively, incubating the nucleic acid molecule with at least one lipid species and at least one protein species to form protein-lipid-polynucleotide complexes consisting essentially of the nucleic acid molecule and the lipid-protein cell-uptake component may form a cell-uptake component. Lipid vesicles made according to Felgner (WO 91/17424) and/or cationic lipidization (WO 91/16024) or other forms for polynucleotide administration (EP 465,529) may also be employed as cell-uptake components. Nucleases may also be used.

Typically, the substrate is coated with recombinase and cell-uptake component simultaneously so that both recombinase and cell-uptake component bind to the substrate; alternatively, a substrate can be coated with recombinase prior to incubation with a cell-uptake component; alternatively, the substrate can be coated with the cell-uptake component and introduced into cells contemporaneously with a separately delivered recombinase (e.g., by targeted liposomes containing one or more recombinase). A substrate of the invention may be conjugated to a cell-uptake component and coated with at least one recombinase and the resulting cell targeting complex contacted with a target cell under uptake conditions (e.g., physiological conditions) so that the substrate and the recombinase(s) are internalized in the target cell. Most preferably, coating of both recombinase and cell-uptake component saturates essentially all of the available binding sites on the substrate. A substrate may be preferentially coated with a cell-uptake component so that the resultant targeting complex comprises, on a molar basis, more cell-uptake component than recombinase(s). Alternatively, a substrate may be preferentially coated with recombinase(s) so that the resultant targeting complex comprises, on a molar basis, more recombinase(s) than cell-uptake component.

Cell-uptake components are included with recombinase-coated targeting polynucleotides of the invention to enhance the uptake of the recombinase-coated targeting polynucleotide(s) into cells, particularly for in vivo gene targeting applications, such as gene therapy to treat genetic diseases, including neoplasia, and targeted homologous recombination to treat viral infections wherein a viral sequence (e.g., an integrated hepatitis B virus (HBV) genome or genome fragment) may be targeted by homologous sequence targeting and inactivated. Alternatively, a substrate may be coated with the cell-uptake component and targeted to cells with a contemporaneous or simultaneous administration of a recombinase (e.g., liposomes or immunoliposomes containing a recombinase, and a vector encoding and expressing a recombinase).

In addition to cell-uptake components, targeting components such as nuclear localization signals may be used, as is known in the art.

Homologous Pairing of Nucleic Acid Molecules Having Chemical Substituents

Also provided is a method whereby at least one exogenous polynucleotide containing a chemical substituent is targeted to a preselected target DNA sequence in an intact living target cell, permitting sequence-specific targeting of chemical substituents such as, for example cross-linking agents, metal chelates (e.g., iron/EDTA chelate for iron catalyzed cleavage), topoisomerases, endonucleases, exonucleases, ligases, phosphodiesterases, photodynamic porphyrins, free-radical generating drugs, chemotherapeutic drugs (e.g., adriamycin or doxirubicin), intercalating agents, base-modification agents, base analogs and modified bases (e.g. containing fluorescent dyes, or affinity tags like biotin or digoxigenin) immunoglobulin chains, oligonucleotides, and other substituents. The methods of the invention can be used to target such a chemical substituent to a preselected target DNA sequence by homologous pairing for various applications, for example: producing sequence-specific strand scission(s), producing sequence-specific chemical modifications (e.g., base methylation or strand cross-linking), producing sequence-specific localization of polypeptides (e.g., topoisomerases, helicases, or proteases), producing sequence-specific localization of polynucleotides (e.g., loading sites for transcription factors and/or RNA polymerase), and other applications.

Thus, in addition to recombinase and optionally cellular uptake components, the nucleic acid molecule may include chemical substituents. A substrate comprising an exogenous nucleic acid molecule that has been modified with appended chemical substituents may be introduced along with recombinase (e.g., RecA) into a metabolically active target cell to homologously pair with a preselected target DNA sequence. In a preferred embodiment, the nucleic acid molecule is derivatized, and additional chemical substituents are attached, either during or after polynucleotide synthesis, and are thus localized to a specific endogenous target sequence where they produce an alteration or chemical modification to a local DNA sequence. Preferred attached chemical substituents include, but are not limited to: cross-linking agents (see Podyminogin et al., 1995 and Podyminogin et al., 1996), nucleic acid cleavage agents, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, labels, base-modification agents, agents which normally bind to nucleic acids such as labels, and the like (see for example Afonina et al., 1996) immunoglobulin chains, and oligonucleotides. Iron/EDTA chelates are particularly preferred chemical substituents where local cleavage of a DNA sequence is desired (Hertzberg et al., 1982; Hertzberg and Dervan, 1984; Taylor et al., 1984; Dervan, 1986). Further preferred are groups that prevent hybridization of the complementary single stranded nucleic acids to each other but not to unmodified nucleic acids; see for example Kutryavin et al., 1996 and Woo et al., 1996). 2′-O methyl groups are also preferred (see Cole-Strauss et al., 1996; Yoon et al., 1996). Additional preferred chemical substituents include labeling moieties, including fluorescent labels. Preferred attachment chemistries include: direct linkage, e.g., via an appended reactive amino group (Corey and Schultz, 1988) and other direct linkage chemistries, although streptavidin/biotin and digoxigenin/antidigoxigenin antibody linkage methods may also be used. Methods for linking chemical substituents are provided in U.S. Pat. Nos. 5,135,720, 5,093,245, and 5,055,556, which are incorporated herein by reference. Other linkage chemistries may be used at the discretion of the practitioner.

Introduction into Cells

Once the recombinase-substrate compositions, optionally including an isolated extrachromosomal sequence comprising the target DNA, are formulated, they are introduced or administered into target cells. The administration is typically done as is known for the administration of nucleic acids into cells, and, as those skilled in the art will appreciate, the methods may depend on the choice of the target cell. Suitable methods include, but are not limited to, Ca²+-mediated transformation, microinjection, electroporation, lipofection, and the like. By “target cells” herein is meant prokaryotic or eukaryotic cells. Suitable prokaryotic cells include, but are not limited to, bacteria such as E. coli, Bacillus spp., Salmonella spp., Streptomyces spp., and the extremophiles such as thermophilic bacteria, archae and the like. Preferably, the prokaryotic target cells are recombination competent. Suitable eukaryotic cells include, but are not limited to, fungi such as yeast and filamentous fungi, including species of Saccharomyces, e.g., S. cerevisiae, Schizosaccharomyces, e.g., S. pombe, Picchia, Aspergillus, Trichoderma, and Neurospora; plant cells including those of corn, sorghum, tobacco, canola, soybean, cotton, tomato, potato, alfalfa, sunflower, Arabidopsis, wheat and the like; and animal cells, including insects, e.g., Drosophilia, fish, e.g., Fugu rubripes, birds and mammals. Suitable fish cells include, but are not limited to, those from species of salmon, trout, tilapia, tuna, carp, flounder, halibut, swordfish, cod, zebrafish and pufferfish. Suitable bird cells include, but are not limited to, those of chickens, ducks, quail, pheasants and turkeys, and other jungle foul or game birds. Suitable mammalian cells include, but are not limited to, cells from horses, cows, buffalo, swine, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes.

In a preferred embodiment, prokaryotic cells are used. In this embodiment, a preselected target DNA sequence is chosen for alteration. Preferably, the preselected target DNA sequence is contained within an extrachromosomal sequence. By “extrachromosomal sequence” herein is meant a sequence separate from the chromosomal sequences. Preferred extrachromosomal sequences include plasmids (particularly prokaryotic plasmids such as bacterial plasmids), cosmids, phagemids, P1 vectors, viral genomes, yeast, bacterial and mammalian artificial chromosomes (YAC, BAC and MAC, respectively), and other autonomously self-replicating sequences, although this is not required. As described herein, a recombinase and a substrate comprising a pair of nucleic acid molecules comprising targeting polynucleotides which substantially correspond to or are substantially complementary to the target sequence contained on the extrachromosomal sequence, which substrate has at least one single stranded end, are added to the extrachromosomal sequence in vitro. In one embodiment, at least one of the nucleic acid molecules contains at least one nucleotide substitution, insertion or deletion relative to the target DNA sequence. The targeting polynucleotides in the nucleic acid molecules bind to the target DNA sequence in the extrachromosomal sequence to effect homologous recombination and form a recombination intermediate. The intermediate is then introduced into a prokaryotic cell using techniques known in the art. These methods may also be used for eukaryotic cells. In one particular embodiment, the nucleic acid molecules comprise a nucleic acid fragment of interest, the sequence of which does not substantially correspond to or is not substantially complementary to the target sequence, which fragment is positioned between targeting polynucleotides. In this embodiment, targeted homologous recombination results in the insertion of the fragment in the extrachromosomal sequence.

Alternatively, the preselected target DNA sequence is a chromosomal sequence or an extrachromosomal sequence present in the cell. In this embodiment, the nucleoprotein complex(es) comprising recombinase and the substrate is introduced into the target cell. The substrate and the recombinase function to effect homologous recombination, resulting in altered genomic chromosomal or extrachromosomal sequences. Thus, sequences present in a substrate may be inserted into an extrachromosomal sequence or chromosome, as well as employed to delete sequences from an extrachromosomal sequence or chromosome, or replace sequences in an extrachromosomal sequence or chromosome.

In one embodiment, eukaryotic cells are employed which are useful to prepare transgenic non-human animals. Transgenic animals are organisms that contain stably integrated copies of genes or gene constructs in the chromosome which are often derived from genes or portions thereof from another species (a “knock in”) which may replace a gene, or a portion thereof, for instance, the coding region of a gene or a portion thereof, with another gene, e.g., a reporter gene, or may augment the chromosome, or contain deletions of endogenous genes or portions thereof (a “knock out”). Introducing cloned DNA constructs of foreign genes into totipotent cells by a variety of methods, including homologous recombination, can generate these animals. Animals that develop from genetically altered totipotent cells contain the foreign gene or a deletion in the endogenous gene in all somatic cells and also in germ-line cells if the foreign gene was integrated into the genome of the recipient cell before the first cell division. Currently methods for producing transgenics have been performed on totipotent embryonic stem cells (ES) and with fertilized zygotes. ES cells have an advantage in that large numbers of cells can be manipulated easily by homologous recombination in vitro before they are used to generate transgenics. Alternatively, DNA can also be introduced into fertilized oocytes by micro-injection into pronuclei which are then transferred into the uterus of a pseudo-pregnant recipient animal to develop to term. For making transgenic non-human animals (which include homologously targeted non-human animals) embryonal stem cells (ES cells) and fertilized zygotes are preferred.

In a preferred embodiment, non-human zygotes are used, for example to make transgenic animals, using techniques known in the art (see U.S. Pat. No. 4,873,191). Preferred zygotes include, but are not limited to, animal zygotes, including insect, e.g., Drosophilia, fish, avian and mammalian zygotes. Suitable fish zygotes include, but are not limited to, those from species of salmon, trout, tuna, carp, flounder, halibut, swordfish, cod, tilapia, zebrafish and pufferfish. Suitable bird zygotes include, but are not limited to, those of chickens, ducks, quail, pheasant, turkeys, and other jungle fowl and game birds. Suitable mammalian zygotes include, but are not limited to, cells from horses, cows, buffalo, deer, swine, sheep, rabbits, rodents such as mice, rats, hamsters and guinea pigs, goats, pigs, primates, and marine mammals including dolphins and whales (see Hogan et al., 1994).

The vectors containing the DNA segments of interest can be transferred into the host cell by well-known methods, depending on the type of cellular host. For example, microinjection is commonly utilized for target cells, although calcium phosphate treatment, electroporation, lipofection, biolistics or viral-based transfection also may be used. Other methods used to transform mammalian cells include the use of polybrene, protoplast fusion, and others (see, generally, Sambrook et al., 1989). Direct injection of DNA and recombinase and/or recombinase-coated substrate into target cells, such as skeletal or muscle cells also may be used (Wolff et al., 1990).

Targeting of DNA Sequences

The compositions of the invention find use in a number of applications, including the site-directed modification of extrachromosomal sequences, e.g., cloning, or endogenous sequences within any target cell, methods and compositions for diagnosis, treatment and prophylaxis of genetic diseases of animals, particularly mammals, and the creation of transgenic organisms, including transgenic plants and animals (e.g., to produce targeted sequence modification(s) in a non-human animal, particularly a non-human mammal such as a mouse, which create(s) a disease allele, such as a human disease allele, in a non-human animal, as sequence-modified non-human animals harboring such a disease allele may provide useful models of human and veterinary neoplastic and other pathogenic diseases).

Generally, any preselected target DNA sequence, such as a gene sequence, can be altered by homologous recombination (which includes gene conversion) with a substrate of the invention. In one embodiment, a substrate for recombination comprises a nucleic acid molecule comprising a sequence that is not present in the preselected target sequence(s) (i.e., a nonhomologous portion or mismatch) which may be as small as a single mismatched nucleotide, several mismatches, or may span up to about several kilobases or more of nonhomologous sequence. Generally, such nonhomologous portions are flanked on each side by targeting polynucleotides. Nonhomologous portions are used to make insertions, deletions, and/or replacements in a preselected target DNA sequence, e.g., single or multiple nucleotide substitutions in a preselected target DNA sequence, so that the resultant recombined sequence (i.e., a targeted or recombinant sequence) incorporates some or all of the sequence information of the nonhomologous portion of the nucleic acid molecule. Thus, the nonhomologous regions are used to make variant sequences, i.e., targeted sequence modifications. Additions and deletions may be as small as 1 nucleotide or greater than 1 to 4 kilobases or more. In this way, site directed modifications may be done in a variety of systems for a variety of purposes.

In one embodiment, a nucleic acid molecule comprising a targeting polynucleotide is used to repair a mutated sequence of a structural gene by replacing it or converting it to a wild-type sequence (e.g., a sequence encoding a protein with a wild-type biological activity). For example, such applications could be used to convert a sickle cell trait allele of a hemoglobin gene to an allele which encodes a hemoglobin molecule that is not susceptible to sickling, by altering the nucleotide sequence encoding the beta subunit of hemoglobin so that the codon at position 6 of the beta subunit is converted from Val to Glu (Shesely et al., 1991). Replacing, inserting, and/or deleting sequence information in a disease allele using appropriately selected nucleic acid molecules can correct other genetic diseases, either partially or totally. For example, but not for limitation, a deletion in the human CFTR gene can be corrected by targeted homologous recombination employing a RecA-coated substrate of the invention.

For many types of in vivo gene therapy to be effective, a significant number of cells must be correctly targeted, with a minimum number of cells having an incorrectly targeted recombination event. To accomplish this objective, the combination of: (1) a substrate, (2) a recombinase (to provide enhanced efficiency and specificity of correct homologous sequence targeting), and (3) a cell-uptake component (to provide enhanced cellular uptake of the nucleic acid molecules), provides a means for the efficient and specific targeting of cells in vivo, making in vivo homologous sequence targeting, and gene therapy, practicable.

Several disease states may be amenable to treatment or prophylaxis by targeted alteration of hepatocytes in vivo by homologous gene targeting. For example and not for limitation, the following diseases, among others not listed, are expected to be amenable to targeted gene therapy: hepatocellular carcinoma, HBV infection, familial hypercholesterolemia (LDL receptor defect), alcohol sensitivity (alcohol dehydrogenase and/or aldehyde dehydrogenase insufficiency), hepatoblastoma, Wilson's disease, congenital hepatic porphyrias, inherited disorders of hepatic metabolism, ornithine transcarbamylase (OTC) alleles, HPRT alleles associated with Lesch Nyhan syndrome, etc. Where targeting of hepatic cells in vivo is desired, a cell-uptake component consisting essentially of an asialoglycoprotein-poly-L-lysine conjugate is preferred. The targeting complexes of the invention which may be used to target hepatocytes in vivo take advantage of the significantly increased targeting efficiency produced by association of a substrate with a recombinase which, when combined with a cell-targeting method such as that of WO 92/05250 and/or Wilson et al. (1992) provide a highly efficient method for performing in vivo homologous sequence targeting in cells, such as hepatocytes.

In another embodiment, the methods and compositions of the invention are used for gene inactivation. That is, in addition to correcting disease alleles, exogenous nucleic acid molecules can be used to inactivate, decrease or alter the biological activity of one or more genes in a cell (or transgenic nonhuman animal). This finds particular use in the generation of animal models of disease states, or in the elucidation of gene function and activity, similar to “knock out” experiments. These techniques may be used to eliminate a biological function; for example, a galT gene (alpha galactosyl transferase genes) associated with the xenoreactivity of animal tissues in humans may be disrupted to form transgenic animals (e.g., pigs) to serve as organ transplantation sources without associated hyperacute rejection responses, or eliminate a gene associated with pathogenicity, e.g., in a prokaryote. Alternatively, the biological activity of the wild-type gene may be either decreased, or the wild-type activity altered, for example, to mimic disease states or overexpress a useful protein, e.g., insulin. This includes genetic manipulation of non-coding gene sequences that affect the transcription of genes, including, promoters, repressors, enhancers and transcriptional activating sequences.

Once the specific target genes to be modified are selected, their sequences may be scanned for possible disruption sites (convenient restriction sites, for example). Plasmids are engineered to contain an appropriately sized gene sequence with a deletion or insertion in the gene of interest and at least one flanking region comprises targeting polynucleotides which substantially correspond or are substantially complementary to a target DNA sequence. Vectors containing a targeting polynucleotide sequence are typically grown in E. coli and then isolated using standard molecular biology methods, or may be synthesized as oligonucleotides. Direct targeted inactivation which does not require vectors may also be done. When using microinjection procedures it may be preferable to use a transfection technique with linearized sequences containing only modified target gene sequence and without vector or selectable sequences. The modified gene site is such that a homologous recombinant between the exogenous nucleic acid molecule and the endogenous DNA target sequence can be identified, e.g., by carefully choosing primers and PCR, followed by analysis to detect if PCR products specific to the desired targeted event are present (Erlich et al., 1991).

In addition, the methods of the present invention are useful to add exogenous DNA sequences, such as exogenous genes or extra copies of endogenous genes, to an organism. As for the above techniques, this may be done for a number of reasons, including: to alleviate disease states, for example by adding one or more copies of a wild-type gene or add one or more copies of a therapeutic gene; to create disease models, by adding disease genes such as oncogenes or mutated genes or even just extra copies of a wild-type gene; to add therapeutic genes and proteins, for example by adding tumor suppressor genes such as p53, Rb1, Wt1, NF1, NF2, and APC, or other therapeutic genes; to make superior transgenic animals, for example superior livestock; or to produce gene products such as proteins, for example for protein production, in any number of host cells. Suitable gene products include, but are not limited to, Rad51, alpha-antitrypsin, antithrombin III, alpha glucosidase, collagen, proteases, viral vaccines, tissue plasminogen activator, monoclonal antibodies, Factors VIII, IX, and X, glutamic acid decarboxylase, hemoglobin, prostaglandin receptor, lactoferrin, calf intestine alkaline phosphatase, CFTR, human protein C, porcine liver esterase, urokinase, and human serum albumin.

Thus, in one preferred embodiment, the targeted sequence modification creates a novel sequence that has a biological activity or encodes a polypeptide having a biological activity. In a preferred embodiment, the polypeptide is an enzyme with enzymatic activity.

In a preferred embodiment, the compositions and methods of the invention are useful in site-directed mutagenesis techniques to create any number of specific or random changes at any number of sites or regions within a target sequence (either nucleic acid or protein sequence), similar to traditional site-directed mutagenesis techniques such as cassette mutagenesis and PCR mutagenesis. Thus, for example, the techniques and compositions of the invention may be used to generate site specific variants in any number of systems, including E. coli, Bacillus, Archebacteria, Thermus, yeast (Saccharomyces and Pichia), insect cells (Spodoptera, Trichoplusia, Drosophila), Xenopus, rodent cell lines including CHO, NIH 3T3 and primate cell lines including COS, or human cells, including HT1080 and BT474, which are traditionally used to make variants. The techniques can be used to make specific changes, or random changes, at a particular site or sites, within a particular region or regions of the sequence, or over the entire sequence.

In this and other embodiments, suitable target sequences include nucleic acid sequences encoding therapeutically or commercially relevant proteins, including, but not limited to, enzymes (proteases, recombinases, lipases, kinases, carbohydrases, isomerases, tautomerases, nucleases etc.), hormones, receptors, transcription factors, growth factors, cytokines, globin genes, immunosuppressive genes, tumor suppressors, oncogenes, complement-activating genes, milk proteins (casein, alpha-lactalbumin, beta-lactoglobulin, bovine and human serum albumin), immunoglobulins, milk proteins, pharmaceutical proteins and vaccines, as well as other desirable targets.

Libraries for Genetic Diversity

A preferred embodiment utilizes the methods of the present invention to create novel genes and gene products. Thus, fully or partially random alterations can be incorporated into genes to form novel genes and gene products, to rapidly and efficiently produce a number of new products which may then be screened, as will be appreciated by those in the art.

Thus, the methods of the invention are useful to generate pools or libraries of variant nucleic acid sequences, and cellular libraries containing the variant libraries. In this embodiment, a plurality of substrates of the invention is used. Each substrate comprises a pair of nucleic acid molecules comprising targeting polynucleotides that substantially correspond to or are substantially complementary to a target sequence. Relative to the other member of the pair, at least one member of the pair has at least one single stranded end that is capable of binding recombinase, and relative to the target sequence, the targeting polynucleotides comprise at least one mismatch. The substrate may be generated by endonuclease, e.g., Dnase I, treatment of a population of DNA molecules, e.g., genomic DNA from one species, or structurally related sequences, e.g., a gene family. The substrate may also be generated synthetically using DNA oligonucleotide synthesis processes known to the art.

The plurality of substrates preferably comprises a pool or library of mismatches over some region(s) or all of the entire targeting sequence. However, the variant nucleic acid molecules may each comprise only one or a few mismatches (less than 10) in the targeting sequence. Thus, for example, a pool of degenerate variant nucleic acid molecules is generated, each of which variant nucleic acid molecule comprises one or more mismatches in the targeting polynucleotide(s) relative to the sequence of a reference sequence, for instance, the pool comprises mismatches at 0.01%, 0.1%, 1%, 10%, 30% or more, e.g., 40% up to 100% of the positions in the reference sequence. Moreover, any particular variant nucleic acid molecule in the pool may comprise only one mismatch, or may comprise mismatches at more than one position, for example, at 0.01%, 0.1%, 10%, 30% or more, including 40% up to 100% of the positions. Thus, the plurality of substrates comprises a pool of random and preferably degenerate mismatches.

As will be appreciated by those in the art, the introduction of a pool of variant nucleic acid molecules (in combination with recombinase) to a target sequence, either in vitro to an extrachromosomal sequence or in vivo to a chromosomal or extrachromosomal sequence, can result in a large number of homologous recombination reactions occurring over time. That is, any number of homologous recombination reactions can occur on a single target sequence, to generate a wide variety of single and multiple mismatches within a single target sequence, and a library of such variant target sequences, most of which will contain mismatches and be different from other members of the library. This thus works to generate a library of mismatches.

In one embodiment, the variant nucleic acid molecules are made to a particular region or domain of a sequence (i.e., a nucleotide sequence that encodes a particular protein or protein domain). For example, it may be desirable to generate a library of all possible variants of a binding domain of a protein, without affecting a different biologically functional domain. Thus, the methods of the present invention find particular use in generating a large number of different variants within a particular region of a sequence, similar to cassette mutagenesis but not limited by sequence length. In addition, two or more regions may also be altered simultaneously using these techniques. Suitable domains include, but are not limited to, kinase domains, nucleotide-binding sites, DNA binding sites, signaling domains, receptor binding domains, transcriptional activating regions, promoters, origins, leader sequences, terminators, localization signal domains, and, in immunoglobulin genes, the complementarity determining regions (CDR), Fc, V_{H} and V_{L}.

Thus, for example, the methods of the invention may be used to create superior recombinant reporter genes such as lacZ and green fluorescent protein (GFP); superior antibiotic and drug resistance genes; superior recombinase genes; superior recombinant vectors; and other superior recombinant genes and proteins, including immunoglobulins, vaccines or other proteins with therapeutic value. For example, targeting polynucleotides containing any number of alterations may be made to one or more functional or structural domains of a protein, and then the products of homologous recombination evaluated.

Once made and administered to target cells, the target cells may be screened to identify a cell that contains the targeted sequence modification. This will be done in any number of ways, and will depend on the target gene and nucleic acid molecules as will be appreciated by those in the art. The screen may be based on phenotypic, biochemical, genotypic, or other functional changes, depending on the target sequence. In an additional embodiment, as will be appreciated by those in the art, selectable markers or marker sequences may be included in the nucleic acid molecules to facilitate later identification. Alternatively, a negative (or counter) selectable marker, such as galK, a suppressor, HSV tK, gpt, URA3, sacB, ccdB, tet^{R}, or 5FOA gene, may be employed to select against certain events, e.g., non-targeted recombinants. If selection is employed, subsequent targeting of the selectable gene via homologous recombination may be used to remove, replace or otherwise disrupt the gene.

In a preferred embodiment, kits containing reagents for homologous recombination and optionally comprising substrates of the invention, are provided. The kits may include recombinases, other enzymes such as exonuclease III, polymerase such as T4 DNA polymerase, helicase, lambda exonuclease, T7 gene 6, DNase I, buffers, dATP and/or ATPyS, and the like.

The invention will be further described by the following non-limiting examples.

### EXAMPLE I

### E. coli RecA and Thermotoga RecA Coating Reactions

RecA is a DNA dependent ATPase that binds cooperatively to single stranded DNA (ssDNA) and double stranded DNA (dsDNA), and promotes homologous pairing and DNA strand exchange between homologous DNA molecules. To purify Thermotoga RecA, Thermotoga maritima DNA was obtained from ATCC and the gene for RecA cloned using the genome sequence available from the NCBI (National Center for Biotechnology Information). E. coli containing the recombinant Thermotoga RecA clone was heated at 65° C., then the heated mixture was sequentially precipitated with PEI (polyethylenimine) and ammonium sulfate. The precipitate was passed over a hydroxyapatite column, a heparin sepharose column, a phosphocellulose column and a Q concentration column. With the exception of the initial heat denaturization step, E. coli RecA may be similarly purified. To characterize a purified recombinase preparation, standard activity assays, e.g., strand exchange, nucleoprotein assembly (see below), or ATPase activity, as well as standard contaminant assays, for instance, a DNase assay, can be employed.

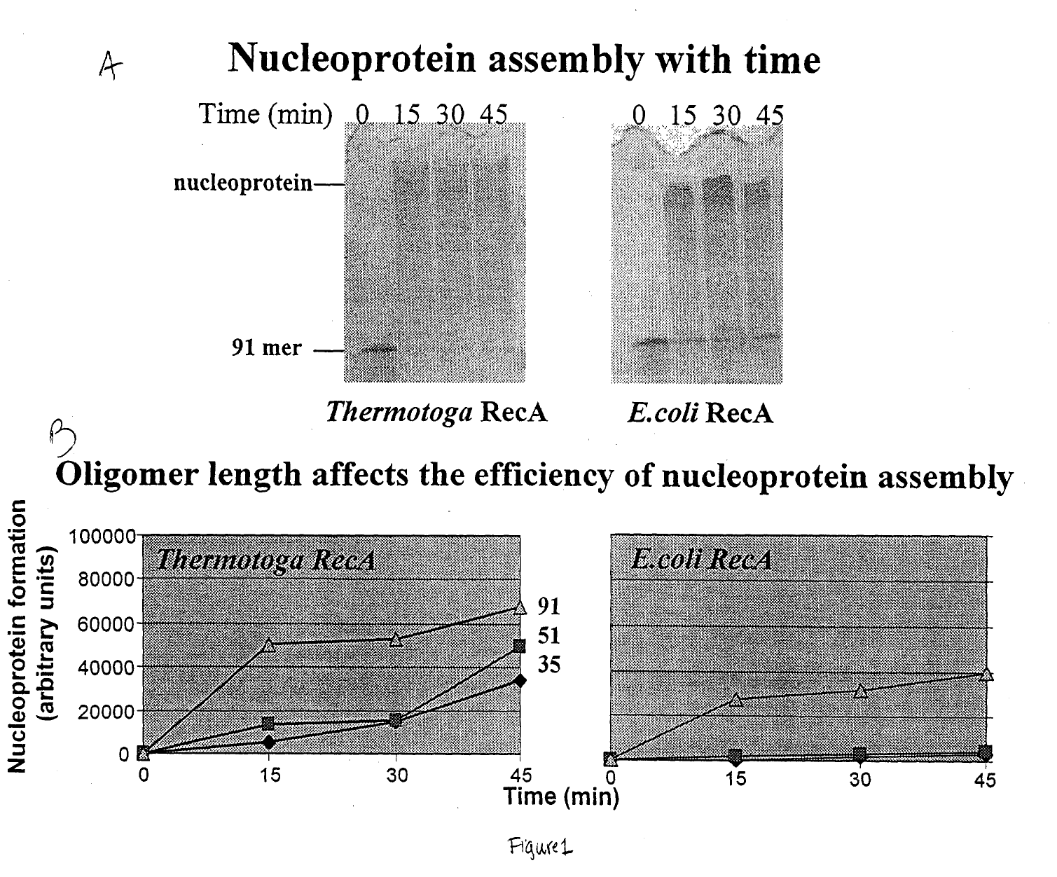

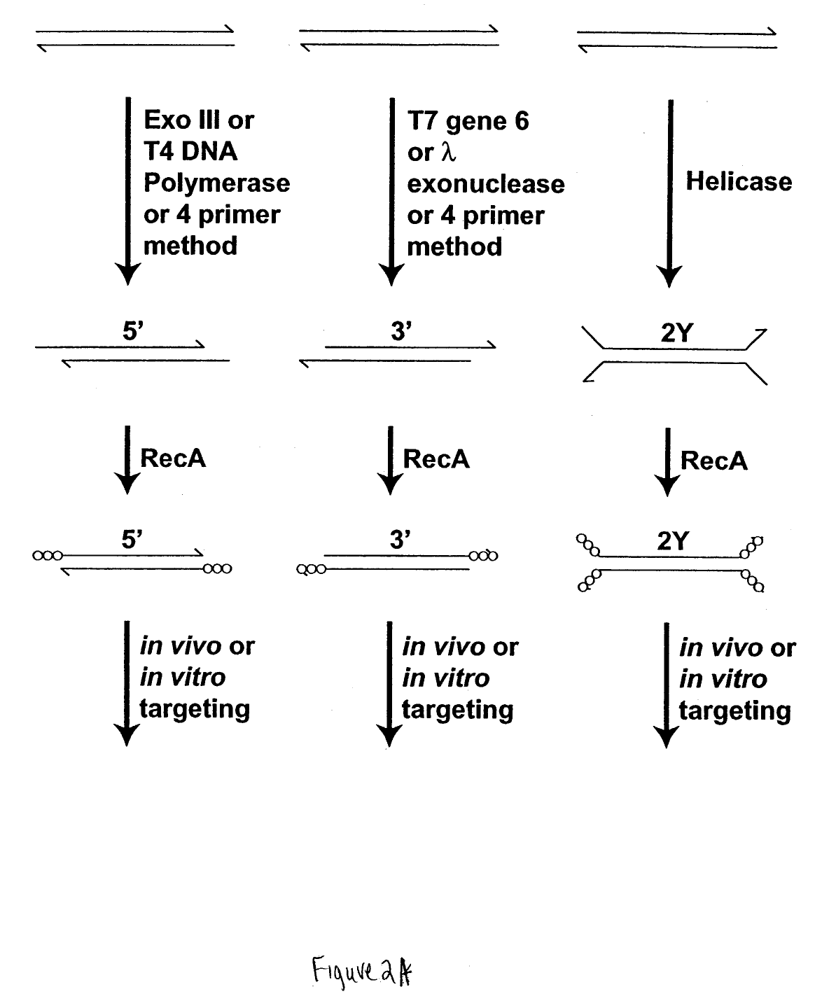

To detect recombinase coating of a substrate (nucleoprotein assembly), a gel shift assay with a labeled ssDNA may be employed. For example, 0.1 μM of a fluorescein-tagged 91-mer oligonucleotide (F-ACAACAACTGGCGGGCAAACAGTCGTTGCTGATTGGC GTTGCCTAATCCAGTCTGGCCCTGCACGCGCCGTCGCAAATTGTCGCGGC GAT; SEQ ID NO: 1) was used as a substrate for coating by RecA. The coating buffer for E. coli RecA was 25 mM Tris acetate, pH 7.85, 15 mM potassium glutamate (K-Glu), 5 mM Mg acetate, and 2.5 mM DTT. The coating buffer for Thermotoga RecA was 25 mM Tris acetate, pH 8.0, 15 mM K-Glu, 2 mM Mg acetate, 2.5 mM DTT and 0.1% Triton. The coating buffer also included ATPyS (3 mM), or dATP and ATPγS at a ratio of 10:1 (3 mM and 0.3 mM, respectively). RecA was then added to the coating buffer containing the substrate at a ratio of 4 μM RecA for 10 μM of base. The E. coli RecA coating reaction was incubated for up to 60 minutes at 42° C. and the Thermotoga RecA coating reaction was incubated for up to 60 minutes at 75° C. (or 65° C.), although other temperatures may be employed. Samples taken at 0, 15, 30 and 45 minutes are shown in FIG. 1A. The tagged oligonucleotide was visualized and quantified using a FluorImager-SI and ImageQuant Software.

Three labeled substrates of differing lengths, a 51-mer, 35-mer and 91-mer oligonucleotide substrate (F-CAGTCGTTGCTGATTGGCGTTGCCTAATCCAGTCTGGCCCTGCACGCGCC G; SEQ ID NO:2, F-GCTGATTGGCGTTGCCTAATCCAGTCTGGCCCTGC; SEQ ID NO:3, F-ACAACAACTGGCGGGCAAACAGTCGTTGCTGATTGGC GTTGCCTAATCCAGTCTGGCCCTGCACGCGCCGTCGCAAATTGTCGCGGC GAT; SEQ ID NO: 1, respectively), at a concentration of 10 μM base, were each mixed with 4 μM Thermotoga RecA in Thermotoga RecA coating buffer or with 4 μM E. coli RecA in E. coli coating buffer. The mixtures were incubated at 75° C. (for Thermotoga RecA) or 42° C. (for E. coli RecA) for up to 60 minutes. Samples taken at 0, 15, 30 and 45 minutes are shown in FIG. 1B. The results showed that nucleoprotein assembly with shorter oligonucleotides, for example the 35-mer and the 51-mer, was slower than assembly with a longer oligonucleotide (FIG. 1B). Also, Thermotoga RecA was more efficient at assembly with all of the substrates relative to E. coli Rec A (FIG. 1B).

### EXAMPLE II

### Recombination Efficiencies with a Partially ssDNA Substrate with 5′ Staggered Ends and a Partially ssDNA Substrate with 3′ Staggered Ends

The tet^{R} gene was chosen as a substrate for recombination with the target pGEM110 (a derivative of pGEM11zf+, Promega Corporation). Primers employed in a PCR for substrate preparation are shown in Table I.

TABLE I

| | | |
|---|---|---|
| Primer | Sequence 5′ to 3′ | Comments |

| | | | |
|---|---|---|---|
| 14705 | HO-AGTCGGCCGAGCTCGAATT; | SEQ ID NO:4 | Upper primer- |
| | | | targeting |
| | | | polynucleotide |
| 14706 | HO-AGCTTATGCATGCGGCCGC; | SEQ ID NO:5 | Lower primer- |
| | | | targeting |
| | | | polynucleotide |
| 14951 | BtnTEG-AGCTTATGCATGCGGCCGC; | SEQ ID NO:6 | Lower primer- |
| | | | targeting |
| | | | polynucleotide |
| 14952 | BtnTEG-AGTCGGCCGAGCTCGAATT; | SEQ ID NO:7 | Upper primer- |
| | | | targeting |
| | | | polynucleotide |
| 15181 | Fluorescein-AGTCGGCCGAGCTCGAATT; | SEQ ID NO:8 | Probe |
| | | | homologous to |
| | | | 3′ ssDNA |
| | | | ends |
| 15182 | Fluorescein-AGCTTATGCATGCGGCCGC; | SEQ ID NO:9 | Probe |
| | | | homologous to |
| | | | 3′ ssDNA |
| | | | ends |
| 15321 | HO-CCGAGATGCGCCGCGTGC; | SEQ ID NO:10 | Lower primer- |
| | | | tet^{R} gene |
| 15320 | HO-AATTCTCATGTTTGATTTGACAGCTTATCAT; | SEQ ID NO:11 | Upper primer- |
| | | | tet^{R} gene |
| 17895 | BtnTEG-AATTCTCATGTTTGATTTGACAGCTTATCAT; | SEQ ID NO:12 | Upper primer- |
| | | | tet^{R} gene |
| 17892 | BtnTEG-CCGAGATGCGCCGCGTGC; | SEQ ID NO:13 | Lower primer- |
| | | | tet^{R} gene |
| 15995 | AATTCGAGCTCGGCCGACTT-Fluorescein; | SEQ ID NO:14 | Probe |
| | | | homologous to |
| | | | the 5′ ssDNA |
| | | | ends |
| 15997 | GCGGCCGCATGCATAAGCTT-Fluorescein; | SEQ ID NO:15 | Probe |
| | | | homologous to |
| | | | the 5′ ssDNA |
| | | | ends |

The PCR product anticipated using the tet^{R} gene as a template with various biotinylated primer pairs is shown in Table II. The PCR conditions were 2 minutes at 95° C., with 42 cycles of: 30 seconds at 95° C., 30 seconds at 60° C., and 1.2 minutes at 72° C., followed by 10 minutes at 72° C. The PCR reaction mixture included primers (2 pmol), 0.2 mM dNTPs, 0.1 U Pfu cloned polymerase (Stratagene) and 0.2 μl of template in 1×PCR buffer. The PCR reaction was followed by Wizard direct purification (Promega Corporation).

TABLE II

| | | PCR | | |
|---|---|---|---|---|
| | Primers | product | Length | Biotinylated |
| DNA fragment | (Position) | location | bp | residue |
| Biotinylated dsDNA | 14952 (1-19) | 1-1552 | 1552 | 1 and 1552 |
| fragment | 14951 (1552- | | | |
| | 1534) | | | |
| Biotinylated | 14952 (1-19) | 1-1490 | 1490 | 1 |
| fragment 1 for | 15321 (1490- | | | |
| substrate with 3′ | 1473) | | | |
| staggered ends | | | | |
| Biotinylated | 15320 (60-85) | 60-1552 | 1492 | 1552 |
| fragment 2 for | 14951 (1552- | | | |
| substrate with 3′ | 1534) | | | |
| staggered ends | | | | |
| Biotinylated | 17895 (60-85) | 60-1552 | 1492 | 60 |
| fragment 3 for | 14706 (1552- | | | |
| substrate with 5′ | 1534) | | | |
| staggered ends | | | | |
| Biotinylated | 14705 (1-19) | 1-1490 | 1490 | 1490 |
| fragment 4 for | 17892 (1490- | | | |
| substrate with 5′ | 1473) | | | |
| staggered ends | | | | |

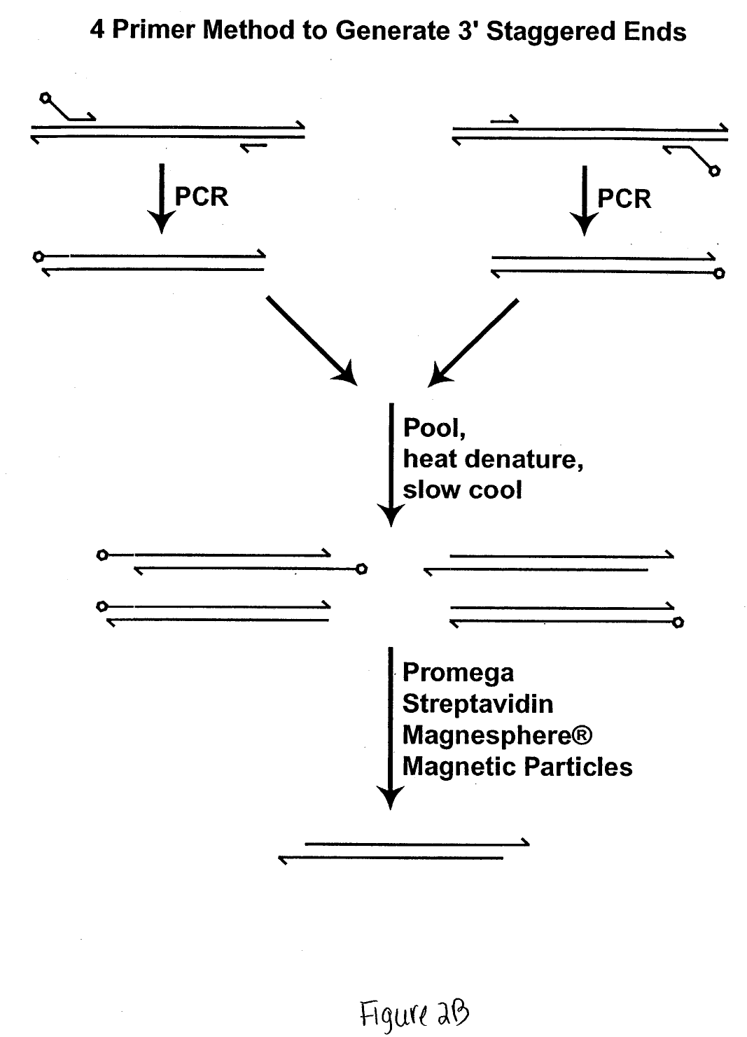

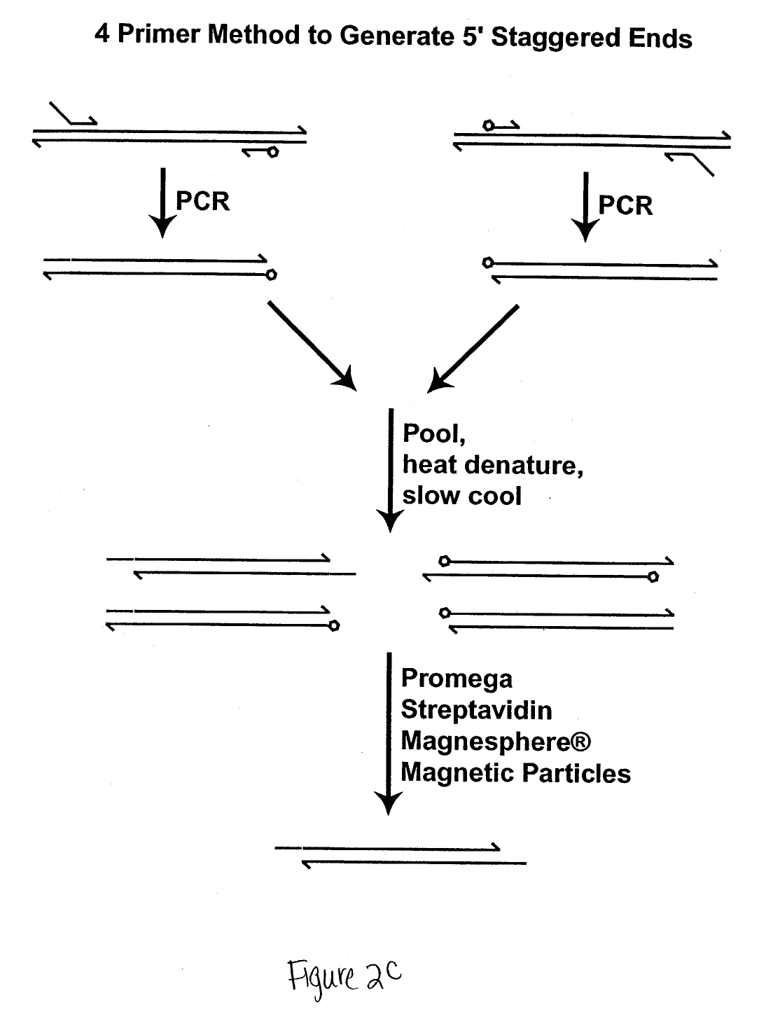

To prepare substrates comprising DNA molecules with 5′ or 3′ staggered ends (FIG. 2A), an equimolar amount of both purified PCR fragments (see Table II, fragments 1 and 2 for the substrate with 3′ staggered ends and fragments 3 and 4 for the substrate with 5′ staggered ends) was mixed then boiled for 5 minutes. The mixture was then cooled gradually to room temperature yielding a partially ssDNA substrate with a ds region of 1430 nucleotides and a ss region of 60 nucleotides at the 5′ or 3′ end. For magnetic separation (see FIG. 2B), streptavidin-magnetic beads were resuspended, 30 μl of beads were placed in a fresh tube and the storage buffer removed using a magnetic stand. The beads were washed three times with 100 μl of binding buffer (1 mM EDTA, 10 mM Tris-HCl, pH 7.5, and 1 M NaCl) by vortexing gently and removing the supernatant with the magnetic stand. The beads were then resuspended in 30 μl of binding buffer for each 15 μl of beads. Nonspecific binding sites on the beads were saturated by adding 5 μg of herring sperm DNA and this mixture was incubated with occasional shaking at room temperature for 10 minutes. The supernatant was removed using the magnetic stand and the beads resuspended with the same volume of binding buffer.

To capture the biotinylated molecules, the beads were transferred to the reaction tube, mixed gently and incubated at room temperature for 30 minutes with rotation. The unbounded DNA was then transferred to a new tube with fresh beads and incubated for another 30 minutes at room temperature. The unbounded DNA (a partially ssDNA substrate with staggered ends) was transferred to a fresh tube and ethanol precipitated.

To verify the structure of the molecules, an annealing reaction was employed between the partially ssDNA fragment with either 5′ or 3′ staggered ends and a fluorescein-tagged oligonucleotide (oligonucleotides 15182 and 15181 for the 3′ overhangs and oligonucleotides 15997 and 15995 for the 5′ overhangs). The tagged structure was run on a 5% acrylamide gel and visualized with a fluorescence scanner.

Coating reactions (4 μM RecA: 10 μM bases) were conducted with a denatured dsDNA substrate (233.4 μM bases), or the partially ssDNA substrates (116.7 μM bases), and 4 mM dATP, 0.08 mM ATPγS and E. coli RecA or Thermotoga RecA in the buffers described above at 42° C. or 75° C., respectively, for 30 minutes. SDS may optionally be added to the loading buffer.

To prepare recombination intermediates for targeting by homologous recombination, the Mg concentration was raised to 12 mM, the target (0.0199 pmol/μl) added (substrate:target ratio of 8:1), and the reactions incubated at 42° C. (E. coli) or 65° C. to 75° C. (Thermotoga) for 60 minutes. Analysis on an agarose gel of 0.5% showed that the stability of the intermediates was: partially ssDNA with 5′ staggered ends>denatured dsDNA>partially ssDNA with 3′ staggered ends. For proteolytic removal of RecA, SDS and proteinase K were added to the reaction at the same time. Since the intermediate is unstable in the absence of RecA, it is unlikely a double D-loop is a significant component of the recombination intermediate.

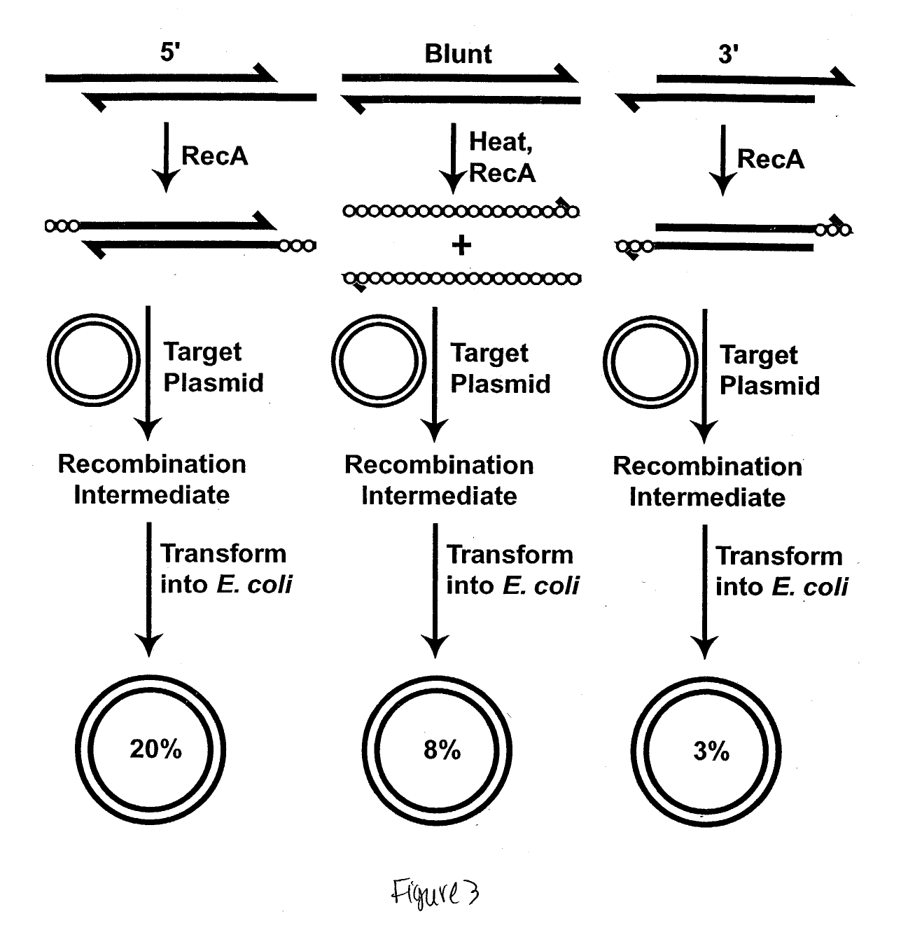

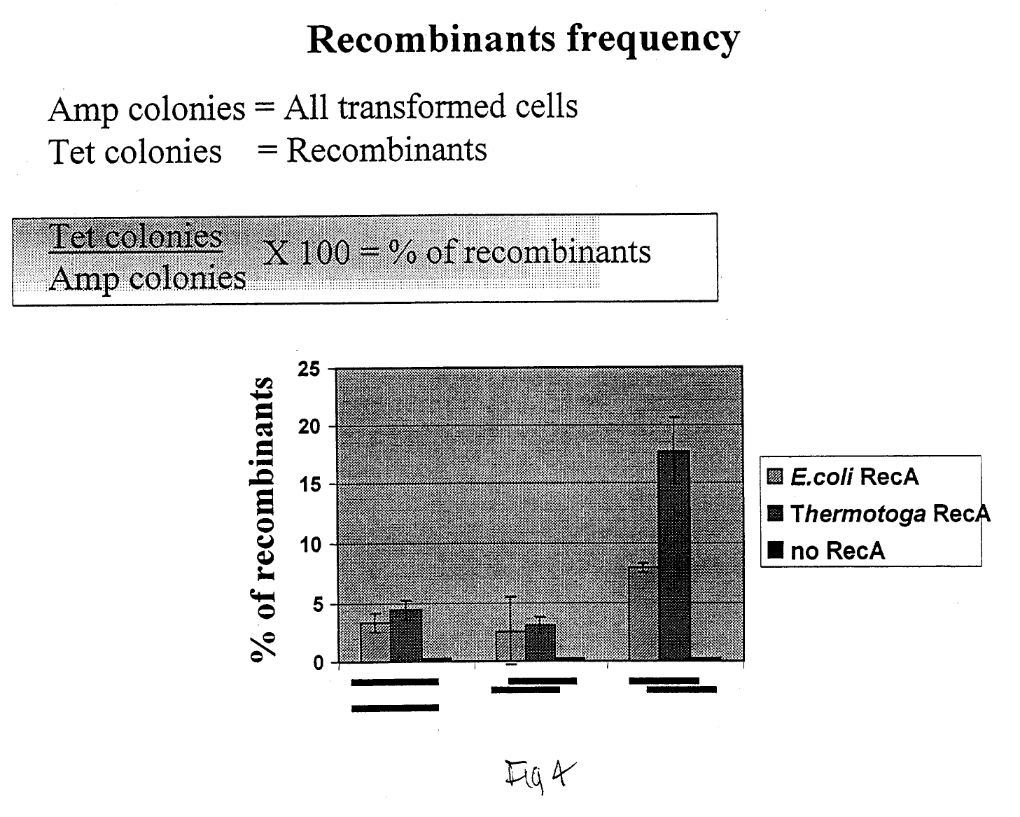

The intermediates were introduced to E. coli strain JC8679 (RecE recombination competent) by electroporation or Ca²⁺ chloride-mediated transformation for in vivo resolution of the recombination intermediates. The stability of the intermediate was found to correlate with the percent of tet^{R} recombinants. The recombination frequency obtained with the partially ssDNA substrate with 5′ staggered ends coated with Thermotoga RecA was 17% (FIG. 3). Moreover, for each RecA tested, the recombination frequency obtained with the partially ssDNA substrate with 5′ staggered ends was at least 2-fold greater than the recombination frequency with the denatured dsDNA substrate. Further, intermediates with Thermotoga RecA yielded a higher percent of recombinants relative to E. coli RecA. The percent of recombinants obtained, e.g., with the partially ssDNA substrate with 5′ staggered ends is sufficiently high that positive selection for recombinants could readily be omitted (FIG. 4). In the absence of RecA, the percent of Tet^{R} recombinants was very low (<0.01%).

### EXAMPLE III

### Recombination Efficiencies with tet^{R} or neo^{R} Substrates with 5′ Staggered Ends or Denatured dsDNA

The efficiency of two different substrates for recombination with a plasmid target, pGEM110, was determined. The substrates included a denatured dsDNA substrate comprising a tet^{R} gene or a neo^{R} gene (1552 bases and 1283 bases, respectively), or a partially ssDNA substrate comprising a tet^{R} gene or a neo^{R} gene and 5′ staggered ends. To prepare the partially ssDNA, equimolar amounts of two dsDNA fragments, each fragment having a biotin affinity tag at one end, were boiled for 5 minutes, gradually cooled, mixed with streptavidin-coated magnetic particles and then subjected to magnetic separation. The structure of the unlabeled DNAs was confirmed using fluorescently labeled oligonucleotides. The dsDNA substrate was heated at 95° C. for 5 minutes followed by 5 minutes on ice.

Coating reactions (40 μl) with the denatured dsDNA substrate and E. coli RecA or Thermotoga RecA (4 μM RecA: 10 μM bases; 923 μM for the tet^{R} gene, and 904 μM for the neo^{R} gene) in coating buffer with 4 mM dATP and 0.08 mM ATPγS were incubated for 30 minutes at 42° C. (E. coli) or 75° C. (Thermotoga). Coating reactions (40 μl) with the partially ssDNA substrate and E. coli RecA or Thermotoga RecA (4 μM RecA:10 μM bases, where μM bases is calculated as ssDNA 227.7 μM for the tet^{R} gene and 245 μM for the neo^{R} gene), in coating buffer with 4 mM dATP and 0.08 mM ATPγS were incubated for 30 minutes at 42° C. (E. coli) or 65° C. (Thermotoga).

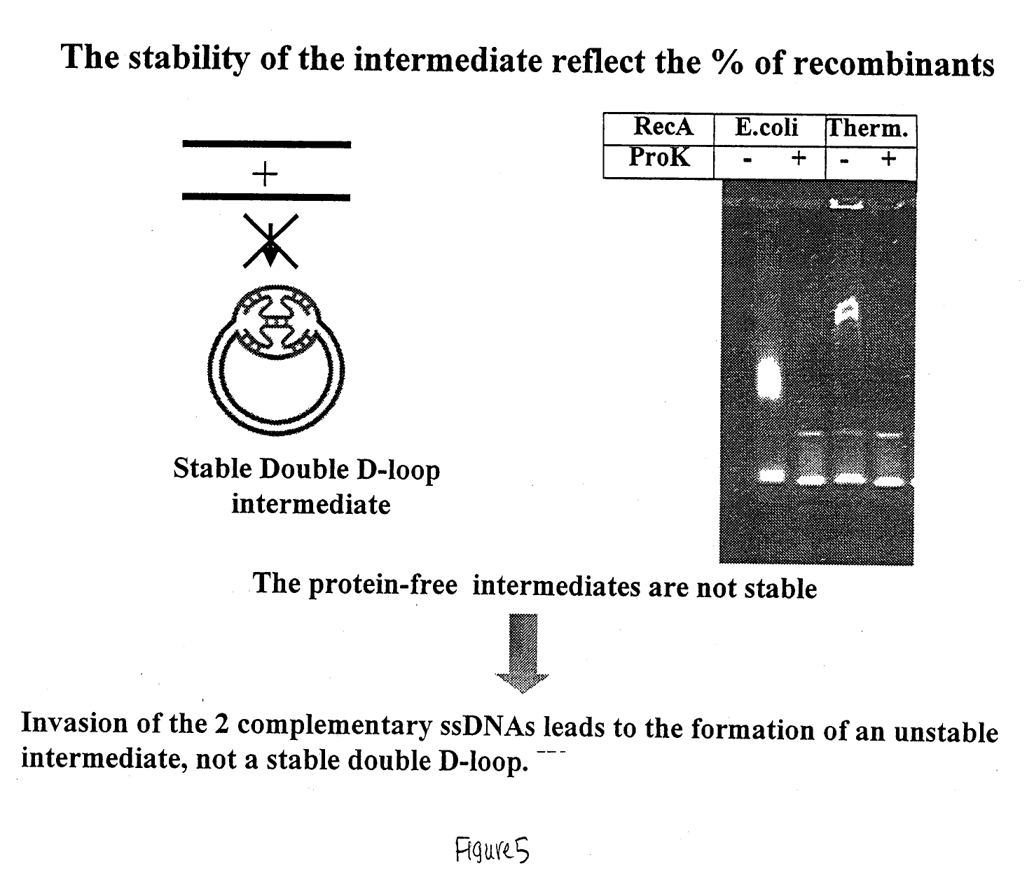

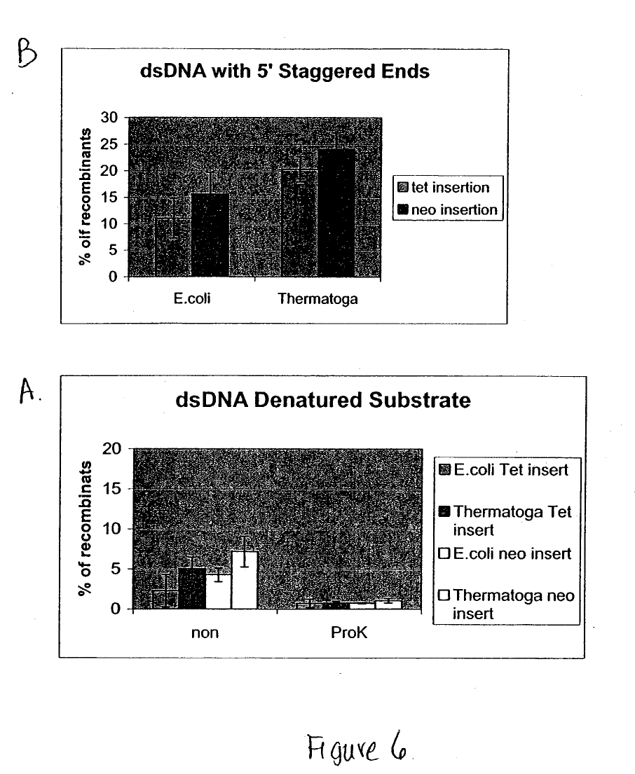

For intermediate formation, the Mg concentration was elevated to 12 mM using Mg acetate, 2.5 μl of target (0.014 pmol/μl; a substrate to target ratio of 6:1) was added and the reaction incubated at 42° C. (E. coli) or 65° C. to 75° C. (Thermotoga) for 60 minutes. After 60 minutes, a portion of the reaction was subjected to proteinase K treatment (200 μg/ml in 2% SDS). It was found that the formation of intermediates with Thermotoga RecA was more efficient than with E. coli RecA, and that intermediates formed with a denatured dsDNA substrate were not stable following proteinase K treatment (it collapsed to the original molecules) (FIG. 5). This would not have been predicted if a double D-loop had formed. The intermediate formation results were supported by the transformation results. The removal of RecA prior to transformation decreased the percent of recombinants by 3- to 7-fold (FIG. 6A). Thermotoga RecA gave approximately 2-fold higher numbers of tet^{R} and neo^{R} recombinants relative to E. coli RecA (FIG. 6). Also, the percent of recombinants with the partially ssDNA substrate was higher (4-fold) relative to the denatured dsDNA substrate (i.e., a double D-loop was not formed for large inserts, resulting in an unstable intermediate).

### REFERENCES

Adzuma, Genes Devel., 6: 1679 (1992).

Afonina et al., PNAS USA, 93: 3199 (1996).

Ausubel et al., “Short Protocols in Molecular Biology,” 2nd ed. (John Wiley & Sons: New York), pp. 9-14 and 9-15 (1992).

Bardwell, Mutagenesis, 4: 245 (1989).

Baumann et al., Cell, 87: 757 (1996).

Beaucage et al., Tetrahedron, 49(10): 1925 (1993).

Behr et al., Proc. Natl. Acad. Sci. USA, 86:6982 (1989).

Berger and Kimmel, Methods in Enzymology. Volume 152. Guide to Molecular Cloning Techniques (1987), Academic Press, Inc., San Diego, Calif.

Berinstein et al., Molec. Cell. Biol., 12: 360 (1992).

Bertling, Bioscience Reports, 7:107 (1987).

Bertolotti, Newsletter of BioTechnology Health and Environmental Sciences, N14 (1996).

Bishop et al., Cell, 69: 439 (1992).

Brinster et al., PNAS, 86:7087 (1989).

Camerini-Otero et al., Annu. Rev. Genetics, 29:509 (1995).

Capecchi, Science, 244:1288 (1989).

Carlsson et al., Nature, 380: 207 (1996).

Cassuto et al., Mol. Gen. Genet., 208: 10 (1987).

Cavard et al., Nucleic Acids Res., 16: 2099 (1988).

Cheng et al., J. Biol. Chem., 263:15110 (1988).

Cheng, et al., NATO ASI Ser., Ser C., Photochemical Probes in Biochemistry, 272:169-177, P. E. Nielsen (ed.), (1989).

Chiu et al., Biochemistry, 32: 13146 (1993).

Clark et al., Molec. Cell. Biol., 11: 2576 (1991).

Cole-Strauss et al., Science, 273:1386 (1996).

Corey and Schultz, Science, 238: 1401 (1988).

Cox et al., Ann. Rev. Biochem, 56:229 (1987).

Cox and Lehman, Ann. Rev. Biochem., 56:229 (1987).

Crameri et al., Nature BioTech., 14:315 (1996).

Crameri et al., Nature Medicine, 2:100-102 (1996).

Cunningham et al., Cell, 24: 213 (1981).

Dervan, P B, Science, 232: 464 (1986).

Doetschman et al., J. Embryol. Exp. Morph., 87: 21 (1985).

Doetschman et al., Proc. Natl. Acad. Sci. (U.S.A.), 85:8583 (1988).

Dorini et al., Science, 243:1357 (1989).

Drumm et al., Cell, 62:1227 (1990).

Dykstra et al., Molec. Cell. Biol., 11: 2583 (1991).

Eckstein, Oligonucleotides and Analogues: A Practical Approach, Oxford University Press).

Egholm, J. Am. Chem. Soc., 114:1895 (1992)

Eisen et al., Proc. Natl. Acad. Sci. USA, 85: 7481 (1988).

Erlich et al., Science, 252: 1643 (1991).

Felgner et al., Proc. Natl. Acad. Sci. USA, 84:7413 (1987).

Ferrin and Camerini-Otero, Science 354:1494 (1991).

Fields and Jang, Science, 249:1046 (1990).

Fishel et al., Proc. Natl. Acad. Sci. (USA), 85: 3683 (1988).

Friedmann, Science, 224: 1275 (1989).

Fu et al., Nucleic Acids Res., 25(3):677 (1997).

Fugisawa et al., Nucl. Acids Res., 13: 7473 (1985).

Ganea et al., Mol. Cell Biol., 7: 3124 (1987).

Gareis et al., Cell. Molec. Biol., 37:191 (1991).

Gates et al., J. Mol. Biol., 255:373 (1996).

Genes, 3rd Ed. (1987) Lewin, B., John Wiley, New York, N.Y.

Haensler and Szoka, Abstract V211 in J. Cell. Biochem. Supplement 16F (1992).

Halbrook et al., J. Biol. Chem., 264: 21403 (1989).

Hasty et al., Molec. Cell. Biol., 11: 5586 (1991).

Hasty et al., Nature, 350:243 (1991).

Herzing et al., Gene, 137:163 (1993).

Hertzberg and Dervan, Biochemistry, 23: 3934 (1984).

Hertzberg et al., J. Am. Chem. Soc., 104: 313 (1982).

Hogan, et al., “Manipulating the Mouse Embryo: A Laboratory Manual”, Cold Spring Harbor Laboratory (1988).

Holliday, Genetic Res., 5:282 (1964).

Hooper et al., Nature, 326: 292 (1987).

Howard-Flanders et al., Nature, 309:215 (1984).

Hsieh et al., Cell, 44: 885 (1986).

Hsieh et al., J. Biol. Chem., 264: 5089 (1989).

Hsieh et al., Genes and Development, 4:1951 (1990).

Hsieh et al, PNAS USA, 89: 6492 (1992).

Hunger-Bertling et al., Mol. and Cellular Biochem., 92:107 (1990). Immunology-A Synthesis, 2nd Edition, E. S. Golub and D. R. Green, Eds., Sinauer Associates, Sunderland, Mass. (1991).

Itzhaki and Porter, Nucl. Acids Res., 19:3835 (1991).

Jasin et al., Proc. Natl. Acad. Sci. USA, 93:8804 (1996).

Jasin and Berg, Genes and Development, 2:1353 (1988).

Jayasena et al., J. Mol. Biol., 230:1015 (1993).

Joyner et al., Nature, 338:153 (1989).

Keene et al., Nucl. Acids Res., 12: 3057 (1984).

Kido et al., Exper. Cell Res., 198:107 (1992).

Kim et al., Gene, 103:227 (1991).

Kim and Smithies, Nucleic Acids Res., 16:8887 (1988).

Kmeic et al., Cold Spring Harbor Symp., 48: 675 (1984).

Kmeic and Hollaman, Cell, 44: 545 (1986).

Koller et al., Proc. Natl. Acad. Sci. (U.S.A.), 88:10730 (1991).

Koller and Smithies, Proc. Natl. Acad. Sci. (U.S.A.), 86:8932 (1989).

Kolodner et al., Proc. Natl. Acad. Sci. USA, 84: 5560 (1987).

Kowalczykowski et al., Microbiol. Rev., 58:401 (1994).

Kowalczykowski et al., Gene Targeting, CRC Press: Boca Raton, ed. Manuel A. Vega, Chap. 7:167 (1995).

Kucherlapati et al., Proc. Natl. Acad. Sci. (U.S.A.), 81:3153 (1984).

Kucherlapati et al., Mol. Cell. Biol., 5:714 (1985).

Kunkel, PNAS USA, 82:488 (1985).

Kunzelmann et al., Gene Therapy, 3:859 (1996).

Langer et al., Proc. Natl. Acad. Sci. USA, 78(11):6633 (1981).

Lavery et al., J. Biol. Chem., 267: 20648 (1992).

Leahy et al., J. Biol. Chem., 26: 954 (1986).

Leahy et al., J. Biol. Chem., 261: 6954 (1986).

Letsinger et al., J. Am. Chem. Soc., 110: 4470 (1988).

Letsinger, J. Org. Chem., 35: 3800 (1970).

Letsinger et al., Nucl. Acids Res., 14: 3487 (1986).

Lewin (ed.), Genes, 3^{rd} ed., John Wiley, New York, N.Y. (1987).

Lopez et al., Nucleic Acids Res., 15:5643 (1987).

Lowenhaupt et al., J. Biol. Chem., 264: 20568 (1989).

Ludwig et al., Soma. Cell and Molecular Genetics, 20(1):11 (1994).

Lukhtanov et al., Nucleic Acids Research, 24(4):683 (1996).

Madiraju et al, Biochem., 31: 10529 (1992).

Madiraju et al., PNAS USA, 85(18): 6592 (1988).

Maniatis et al., Molecular Cloning: A Laboratory Manual (1989), 2nd Ed., Cold Spring Harbor, N.Y.

Mansour et al., Nature, 336:348 (1988).

Matsumura et al., Nature Bio Tech., 14:366 (1996).

McCarthy et al., Proc. Natl. Acad. Sci. USA, 85: 5854 (1988).

McEntee et al., J. Biol. Chem., 256: 8835 (1981).

McMahon and Bradley, Cell, 62: 1073 (1990).

McIlwraith et al., Nucleic Acids Research, 29(22): 4509 (2001)

Meier et al., Chem. Int. Ed. Engl., 31: 1008 (1992).

Meyer, Jr. et al., J. of the Amer. Chem. Soc., 111(22):8517 (1989).

Moore et al., J Biol. Chem., 19: 11108 (1990).

Moore et al., Proc. Natl. Acad. Sci. (U.S.A.) 88: 9067 (1991).

Mortensen et al., Proc. Natl. Acad. Sci. USA, 88: 7036 (1991).

Mouellic et al., Proc. Natl. Acad. Sci. USA, 87: 4712 (1990).

Nielsen, Nature, 365: 566 (1993).

O'Gorman et al., Science, 251:1351 (1991).

Onouchi et al., Nucleic Acids Res., 19: 6373 (1991).

Oppliger et al., Mut. Res., 291:181 (1993).

Orkin et al., National Institutes of Health, Dec. 7, 1995.

Papworth et al., Strategies, 9:3 (1996).

Pati et al., Encycl. of Cancer, vol. III: 1601-1625 (1997).

Pauwels et al., Chemica Scripta, 26: 141 (1986).

Podyminogin et al., Biochem., 34: 13098 (1995).

Podyminogin et al., Biochem., 35: 7267 (1996).

Radding, C. M., Ann. Rev. Genet., 16:405 (1982).

Ramdas et al., J. Biol Chem., 264:11395 (1989).

Rao et al., PNAS, 88:2984 (1991).

Rashid et al., Nucleic Acids Research, 25:719 (1997).

Rawls, C&EN, p.11 (1996).

Register et al., J. Biol. Chem., 262:12812 (1987).

Reid et al., Molec. Cellular Biol., 11: 2769 (1991).

Reiss et al., Proc. Natl. Acad. Sci. USA, 93:3094 (1996).

Revet et al., J. Mol. Biol., 232:779 (1993).

Rigas et al., Proc. Natl. Acad. Sci. (U.S.A.), 83: 9591 (1986).

Robertson et al., Nature, 323: 445 (1986).

Robertson, E. J. in Teratocarcinomas and Embryonic Stem Cells: A Practical Approach. E. J. Robertson, ed. (oxford: IRL Press), p. 71-112 (1987).

Roca, A. I., Crit. Rev. Biochem. Molec. Biol., 25: 415 (1990).

Rose et al., BioTechniques, 10:520 (1991).

Rosenfeld et al., Cell, 68:143 (1992).

Sambrook et al., Molecular Cloning: A Laboratory Manual, Cold Spring Harbor, N.Y. (1989).

Samulski et al., EMBO J., 10:2941 (1991).

Sauer and Henderson, New Biologist, 2:441 (1990).

Sawai et al, Chem. Lett., 805 (1984).

Schwartzberg et al., Science, 246: 799 (1989).

Sena et al., Nature Genetics, 3:365-372 (1993).

Shesely et al., Proc. Natl. Acad. Sci. (U.S.A.), 88:4294 (1991).

Shinohara et al., Cell, 6: 457 (1992).

Shulman et al., Molec. Cell. Biol., 10: 4466 (1990).

Smithies et al., Nature, 317:230 (1985).

Snouwaert et al., Science, 257:1083 (1992).

Song et al., Proc. Natl. Acad. Sci. (U.S.A.) 84:6820 (1987).

Sprinzl et al., Eur. J. Biochem., 81: 579 (1977).

Stasiak et al., Cold Spring Harbor Symp. Quant. Biol., 49:561 (1984).

Stemmer, Nature, 370:389 (1994).

Stemmer, Proc. Natl. Acad. Sci. USA, 91:10747-10751 (1994).

Stemmer et al., Gene, 164:49 (1995).

Strobel et al., Science, 254:1639 (1991).

Sugino et al., Proc. Natl. Acad. Sci. USA, 85: 3683 (1985).

Sung et al., Science, 265:1241 (1994).

Susulic et al., J. Biol. Chem., 49: 29483 (1995).

Tabone et al., Biochemistry, 33(1):375 (1994).

Taylor et al., Tetrahedron, 40: 457 (1984).

Teratocarcinomas and embryonic stem cells: a practical approach, E. J. Robertson, ed., IRL Press, Washington, D.C., 1987.

Thomas et al., Cell, 44:419 (1986).

Thomas and Capecchi, Cell, 51:503 (1987).

Tishkoff et al., Molec. Cell. Biol. 11: 2593.

Valancius and Smithies, Molecular and Cellular Biology, 11 (3):1402 (1991).

Voloshin et al., Science, 272:868 (1996).

Wilson et al., J. Biol. Chem., 267: 963 (1992).

Wolff et al., Science, 247: 1465 (1990).

Woo, et al., Nucleic Acids Res., 24(13):2470 (1996).

Woodbury, et al., Biochemistry, 2(20): 4730 (1983).

Wu et al., The Journal of Biological Chemistry, 264(29):16985 (1989).

Wu et al., J. Biol. Chem., 266: 14338 (1991).

Wu and Wu, Biochemistry, 27: 887 (1988).

Wu and Wu, J. Biol. Chem., 262: 4429 (1987).

Wu and Wu, J. Biol. Chem., 263: 14621 (1988).

Wu and Wu, J. Biol. Chem., 267:12436 (1992).

Yoon et al., Proc. Natl. Acad. Sci. USA, 93:2071 (1996).

Zimmer and Gruss, Nature. 338: 150 (1989).

Zjilstra et al., Nature, 342: 435 (1989).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method for targeting and altering, by homologous recombination, a preselected target nucleic acid sequence in an extrachromosomal sequence, comprising:
a) providing a mixture comprising recombinase and an at least partially single stranded nucleic acid substrate for recombination comprising two nucleic acid molecules, wherein the first and the second nucleic acid molecules each comprise targeting polynucleotides which substantially correspond to or are substantially complementary to the preselected target nucleic acid sequence, wherein the two nucleic acid molecules are capable of forming a partially double stranded molecule with each other, and wherein at least the 5′ end or the 3′ end of the first nucleic acid molecule comprises a nucleotide sequence, the substantial complement of which is not present at the 3′ end or 5′ end of the second nucleic acid molecule, which nucleotide sequence is capable of binding recombinase;
b) contacting the mixture with the extrachromosomal sequence to form a recombination intermediate; and
c) introducing the recombination intermediate into a cell to yield an altered cell comprising a genetically altered extrachromosomal sequence comprising a targeted sequence alteration.

2. A method for targeting and altering, by homologous recombination, a preselected target nucleic acid sequence in an extrachromosomal sequence, comprising:
a) providing a mixture comprising recombinase and a nucleic acid substrate for recombination comprising two nucleic acid molecules which together form a substantially double stranded molecule having single stranded 5′ and 3′ ends, wherein the first and the second nucleic acid molecules each comprise targeting polynucleotides which substantially correspond to or are substantially complementary to the preselected target nucleic acid sequence, and wherein at least one of the single stranded ends is capable of binding recombinase;
b) contacting the mixture with the extrachromosomal sequence to form a recombination intermediate; and
c) introducing the recombination intermediate into a cell to yield an altered cell comprising a genetically altered extrachromosomal sequence comprising a targeted sequence alteration.

3. A method for targeting and altering, by homologous recombination, a preselected target nucleic acid sequence in a cell, comprising:
a) providing a mixture comprising recombinase and an at least partially single stranded nucleic acid substrate for recombination comprising two nucleic acid molecules, wherein the first and the second nucleic acid molecules each comprise targeting polynucleotides which substantially correspond to or are substantially complementary to the preselected target nucleic acid sequence, wherein the two nucleic acid molecules are capable of forming a partially double stranded molecule with each other, and wherein at least the 5′ end or the 3′ end of the first nucleic acid molecule comprises a nucleotide sequence, the substantial complement of which is not present at the 3′ end or the 5′ end of the second nucleic acid molecule, which nucleotide sequence is capable of binding recombinase; and
b) contacting a cell with the mixture to yield an altered cell comprising a targeted sequence alteration.

4. A method for targeting and altering, by homologous recombination, a preselected target nucleic acid sequence in a cell, comprising:
a) providing a mixture comprising recombinase and a nucleic acid substrate for recombination comprising two nucleic acid molecules which together form a substantially double stranded molecule having single stranded 5′ and 3′ ends, wherein the first and the second nucleic acid molecules each comprise targeting polynucleotides which substantially correspond to or are substantially complementary to the preselected target nucleic acid sequence, and wherein at least one of the single stranded ends is capable of binding recombinase; and
b) contacting a cell with the mixture to yield an altered cell comprising a targeted sequence alteration.

5. The method of claim 1 or 3 wherein the 5′ end of the first nucleic acid molecule comprises a nucleotide sequence, the substantial complement of which is not present at the 3′ end of the second nucleic acid molecule and wherein the 5′ end of the second nucleic acid molecule comprises a nucleotide sequence, the substantial complement of which is not present at the 3′ end of the first nucleic acid molecule, which nucleotide sequences are capable of binding recombinase.

6. The method of claim 1 or 3 wherein the 3′ end of the first nucleic acid molecule comprises a nucleotide sequence, the substantial complement of which is not present at the 5′ end of the second nucleic acid molecule and wherein the 3′ end of the second nucleic acid molecule comprises a nucleotide sequence, the substantial complement of which is not present at the 5′ end of the first nucleic acid molecule, which nucleotide sequences are capable of binding recombinase.

7. The method of claim 1 or 3 wherein the partially single stranded nucleic acid substrate is also partially double stranded.

8. The method of claim 2 or 4 wherein the single stranded ends are formed using helicase.

9. The method of claim 3 or 4 wherein the preselected target nucleic acid sequence is an extrachromosomal sequence present in the cell to be altered.

10. The method of claim 3 or 4 wherein the preselected target nucleic acid sequence is a chromosomal sequence of the cell.

11. The method of claim 1 or 3 wherein the nucleic acid substrate comprises two single stranded nucleic acid molecules.

12. The method of claim 1, 2, 3, or 4 wherein the mixture is introduced into the cell by lipofection, transfection, microinjection, electroporation, laser poration, biolistics or calcium-mediated transformation.

13. The method of claim 1, 2, 3, or 4 wherein each targeting polynucleotide is greater than 20 nucleotides in length.

14. The method of claim 1, 2, 3, or 4 wherein at least one of the nucleic acid molecules comprises a plurality of deletions, a plurality of additions, a plurality of substitutions, or any combination thereof, relative to the preselected target nucleic acid sequence.

15. The method of claim 1, 2, 3, or 4 wherein the nucleotide sequence comprises targeting polynucleotides.

16. A method of generating a library of recombination intermediates comprising variant nucleic acid sequences of a preselected target nucleic acid sequence in an extrachromosomal sequence, comprising adding to the extrachromosomal sequence, recombinase and a plurality of at least partially single stranded nucleic acid substrates for recombination, to form a library of recombination intermediates, wherein each substrate comprises two variant nucleic acid molecules, wherein the first and the second variant nucleic acid molecules each comprise targeting polynucleotides which substantially correspond to or are substantially complementary to the preselected target nucleic acid sequence, wherein the two variant nucleic acid molecules are capable of forming a partially double stranded molecule with each other, wherein at least the 5′ end or the 3′ end of the first variant nucleic acid molecule comprises a nucleotide sequence, the substantial complement of which is not present at the 3′ end or 5′ end of the second variant nucleic acid molecule, which nucleotide sequence is capable of binding recombinase, and wherein the plurality of substrates comprise a library of mismatches between the targeting polynucleotides and the target nucleic acid sequence.

17. The method of claim 16 further comprising introducing the library into cells.

18. The method of claim 17 wherein the cells are prokarylotic cells.

19. The method of claim 17 wherein the cells are eukaryotic cells.
